(19) United States
(12) Patent Application Publication
(10) Pub. No.: US 2001/0051654 A1
(43) Pub. Date: Dec. 13, 2001

(54) Treatment of inflammatory and autoimmune diseases

(76) Inventors: Peter J. Elliott, Marlborough, MA (US); Julian Adams, Brookline, MA (US); Louis Plamondon, Watertown, MA (US)

Correspondence Address: HALE AND DORR, LLP, 60 STATE STREET, BOSTON, MA, 02109

(21) Appl. No.: 09/770,889

(22) Filed: Jan. 26, 2001

Related U.S. Application Data
(63) Continuation of application No. 09/393,794, filed on Sep. 10, 1999, now abandoned.
Application No. 09/393,794 is a continuation-in-part of application No. PCT/US98/20065, filed on Sep. 25, 1998.
(60) Provisional application No. 60/061,038, filed on Sep. 25, 1997.
(60) Provisional application No. 60/069,562, filed on Dec. 12, 1997.
(60) Provisional application No. 60/074,887, filed on Feb. 17, 1998.

(51) Int. Cl. A61K 31/365; A61K 31/7048

(52) U.S. Cl. 514/450; 514/28

(57) ABSTRACT

This invention is directed to the treatment of inflammatory and autoimmune diseases by administering proteasome inhibitors, ubiquitin pathway inhibitors, agents that interfere with the activation of NF-κB via the ubiquitin proteasome pathway, or mixtures thereof. The invention is further directed to the treatment of inflammatory and autoimmune diseases by administering an effective combination of a glucocorticoid and a proteasome inhibitor, ubiquitin pathway inhibitor, agent that interferes with the activation of NF-κB via the ubiquitin proteasome pathway, or mixture thereof. Pharmaceutical compositions comprising a combination of a glucocorticoid and a proteasome inhibitor, ubiquitin pathway inhibitor, agent that interferes with the activation of NF-κB via the ubiquitin proteasome pathway, or mixture thereof are also contemplated within the scope of the invention.

### CROSS-REFERENCE TO RELATED APPLICATIONS

This application is a continuation-in-part of PCT/U.S. Pat. No. 98/20065, filed Sep. 25, 1998, which designates the United States and claims priority from U.S. provisional applications 60/061,038, filed Sep. 25, 1997; 60/069,562, filed Dec. 12, 1997; and 60/074,887, filed Feb. 17, 1998.

### BACKGROUND OF THE INVENTION

1. Field of the invention

This invention is directed to compositions and methods for treatment of inflammatory and autoimmune diseases.

2. Summary of the Related Art

Eukaryotic cells contain multiple proteolytic systems, including lysosomal proteases, calpains, ATP-ubiquitin-proteasome dependent pathway, and an ATP- independent nonlysosomal process. The major neutral proteolytic activity in the cytosol and nucleus is the proteasome, a 20 S (700 kDa) particle with multiple peptidase activities. The 20 S complex is the proteolytic core of a 26 S (1500 kDa) complex that degrades or processes ubiquitin-conjugated proteins. Ubiquitination marks a protein for hydrolysis by the 2 S proteasome complex. Many abnormal or short-lived normal polypeptides are degraded by the ubiquitin-proteasome-dependent pathway. Abnormal peptides include oxidant-damaged proteins (e.g., those having oxidized disulfide bonds), products of premature translational termination (e.g., those having exposed hydrophobic groups which are recognized by the proteasome, and stress-induced denatured or damaged proteins (where stress is induced by, e.g., changes in pH or temperature, or exposure to metals). The proteasome also participates in the rapid elimination and post-translational processing of proteins involved in cellular regulation (e.g., cell cycle, gene transcription, and metabolic pathways), intercellular communication, and the immune response (e.g., antigen presentation).

The transcription factor NF-κB is a member of the Rel protein family. The Rel family of transcriptional activator proteins can be divided into two groups. The first group requires proteolytic processing, and includes p105 and p100, which are processed to p50 and p52, respectively. The second group does not require proteolytic processing and includes p65 (Rel A), Rel (c-Rel), and Rel B. NF-κB comprises two subunits, p50 and an additional member of the Rel gene family, e.g., p 65. Unprocessed p105 can also associate with p65 and other members of the Rel family. In most cells, the p50-p65 heterodimer is present in an inactive form in the cytoplasm, bound to IκB-α. The ternary complex can be activated by the dissociation and destruction of IκB-α, while the p65/p105 heterodimer can be activated by processing of p105.

The ubiquitin-proteasome pathway plays an essential role in the regulation of NF-κB activity, being responsible both for processing of p105 to p50 and for the degradation of the inhibitor protein IκB-α. In order to be targeted for degradation by the proteasome, IκB-α must first undergo selective phosphorylation at serine residues 32 and 36, followed by ubiquitination (Chen et al. Genes & Development (1995) 9:1586; Chen et al. Cell (1996) 84:853; Brockman et al. Mol. Cell. Biol. (1995) 15:2809; Brown et al. Science (1995) 267:1485).

Once activated, NF-κB translocates to the nucleus, where it plays a central role in the regulation of a remarkably diverse set of genes involved in the immune and inflammatory responses (Grilli et al., International Review of Cytology (1993) 143:1-62). For example, NF-κB is required for the expression of a number of genes involved in the inflammatory response, such as TNF-α gene and genes encoding the cell adhesion molecules E-selectin, P-selectin, ICAM, and VCAM (Collins, T., Lab. Invest. (1993) 68:499. NF-κB is also required for the expression of a large number of cytokine genes such as IL-2, IL-6, granulocyte colony stimulating factor, and IFN-β. Inducible nitric oxide synthetase is also under regulatory control of NF-κB. Proteasome inhibitors block IκB-α degradation and activation of NF-κB (Palombella et al. WO 95/25533 published Sep. 28, 1995; Traenckner, et al., EMBO J. (1994) 13:5433). Proteasome inhibitors also block TNF-α induced expression of the leukocyte adhesion molecules E-selectin, VCAM-1, and ICAM-1 (Read, et al., Immunity (1995) 2:493).

Cyclins are proteins involved in cell cycle control. The proteasome participates in the degradation of cyclins. Cyclin degradation enables a cell to exit one cell cycle stage (e.g., mitosis) and enter another (e.g., division). There is evidence that cyclin is converted to a form vulnerable to a ubiquitin ligase or that a cyclin-specific ligase is activated during mitosis (Ciechanover Cell (1994) 79:13). Inhibition of the proteasome inhibits cyclin degradation, and therefore inhibits cell proliferation (Kumatori et al. Proc. Natl. Acad. Sci. USA (1990) 87:7071)

The continual turnover of cellular proteins by the ubiquitin-proteasome pathway is also used by the immune system to screen for the presence of abnormal intracellular proteins (Goldberg and Rock Nature (1993) 357:375). In this process, lymphocytes continually monitor small fragments of cell protein that are presented on class I major histocompatibility complex (MHC) molecules. Proteasomes initially degrade proteins to small peptides, most of which are rapidly hydrolyzed to amino acids by cytosolic exopeptidases. But some of these peptides are transported into the endoplasmic reticulum where they bind to MHC molecules and are then transported to the cell surface in a process known as antigen presentation. If the peptides are abnormal (for example, if they are derived from viral proteins), they elicit cell destruction by cytotoxic T cells. Inhibitors that prevent proteasome function have been shown to block the generation of most of the peptides presented on MHC class I molecules (Rock, et al. Cell (1994) 78: 761).

Multiple sclerosis (MS) is an incurable neurological illness that frequently causes chronic disability. MS is the most common demyelinating disease of the human central nervous system and typically affects youth and women more than men. Clinically, the illness is characterized into a relapsing-remitting or chronic progressive stage, although more precisely defined stages exist for research purposes. It tends to follow a highly unpredictable course leading to chronic and sometimes devastating disability. It is widely believed that MS is the result of an autoimmune disorder in a genetically susceptible individual, mediated by autoreactive T cells that migrate into the CNS and initiate the inflammatory demyelinating lesion.

Airway hyperreactivity to a variety of spasmogens and pulmonary inflammation characterized by eosinophilia are pathologies that are characteristic of asthma (Beasely, et al. Am. Rev. Resp. Dis. (1989) 139:806). Asthma is a chronic condition of the airways that involves many types of inflammatory cell and the release of many mediators and neurotransmitters that have multiple effects on the various target cells in the airway. The degree and extent of inflammation in the airway wall are broadly related to the clinical severity of the asthma. The inflammatory response of asthma consists of activation of mast cells resident in the airways, increased numbers of lymphocytes (which are mainly CD4+T lymphocytes) and an infiltration with eosinophils, which appear to degranulate.

There is a need in the art for effective therapies for the treatment of multiple sclerosis or asthma.

### BRIEF SUMMARY OF THE INVENTION

The present invention is directed to methods for treating a patient afflicted with multiple sclerosis or asthma comprising administering to said patient an effective amount of an agent selected from the group consisting of proteasome inhibitors, ubiquitin pathway inhibitors, agents that interfere with the activation of NF-κB via the ubiquitin proteasome pathway, and mixtures thereof.

In certain embodiments of the invention, the agent is a proteasome inhibitor. Preferably, the proteasome inhibitor is selected from the group consisting of peptidyl aldehydes, boronic acids, boronic esters, lactacystin, and lactacystin analogs. In a preferred embodiment, the proteasome inhibitor is lactacystin or a lactacystin analog, more preferably lactacystin, clasto-lactacystin β-lactone, 7-ethyl-clasto-lactacystin β-lactone, 7-n-propyl-clasto-lactacystin β-lactone, or 7-n-butyl-clasto-lactacystin β-lactone. Most preferably, the proteasome inhibitor is 7-n-propyl-clasto-lactacystin β-lactone.

In other embodiments of the invention, the agent is a ubiquitin pathway inhibitor.

In yet other embodiments of the invention, the agent is one that interferes with the activation of NF-κB by the ubiquitin-proteasome pathway. Preferably the agent that interferes with the activation of NF-κB is an agent that inhibits phosphorylation of IκB-α.

The invention is further directed to methods for treating a patient afflicted with asthma comprising administering to said patient an effective combination of a glucocorticoid and an agent selected from the group consisting of proteasome inhibitors, ubiquitin pathway inhibitors, agents that interfere with the activation of NF-κB via the ubiquitin proteasome pathway, and mixtures thereof.

In a preferred embodiment, the combination comprises an amount of the agent sufficient to reduce the dose or treatment frequency required for the glucocorticoid. In certain preferred embodiments, the combination comprises an amount of the glucocorticoid that is less than its standard recommended dosage. In another preferred embodiment, the combination comprises an amount of the glucocorticoid sufficient to reduce the dose or treatment frequency required for the agent.

In certain preferred embodiments, the glucocorticoid is selected from the group consisting of flunisolide, triamcinolone acetonide, beclomethasone dipropionate, dexamethasone sodium phosphate, fluticasone propionate, budesonide, hydrocortisone, prednisone, prednisolone, mometasone, tipredane, and butixicort.

In other preferred embodiments, the combination used to treat a patient afflicted with asthma comprises a glucocorticoid and a proteasome inhibitor. More preferably, the proteasome inhibitor is lactacystin or a lactacystin analog. Most preferably the combination comprises 7-n-propyl-clasto-lactacystin β-lactone and budesonide.

The invention is further directed to pharmaceutical compositions comprising a combination of a glucocorticoid and an agent selected from the group consisting of proteasome inhibitors, ubiquitin pathway inhibitors, agents that interfere with the activation of NF-κB via the ubiquitin proteasome pathway, or mixtures thereof. In certain embodiments, the pharmaceutical composition is provided in a unit dosage form. Preferably the unit dosage form comprises the glucocorticoid in an amount that is less than its standard recommended dosage.

### BRIEF DESCRIPTION OF THE DRAWINGS

FIG. 1 is a graphical representation of mean clinical score as a function of time in an experimental autoimmune encephalomyelitis model. These data demonstrate that 3b (7-n-propyl-clasto-lactacystin β-lactone) treatment causes a reduction in relapse rate and in mean clinical score as compared to vehicle-treated animals.

FIG. 2 is a graphical representation of relapse rate as a function of time in an experimental autoimmune encephalomyelitis model. These data demonstrate that 3b treatment causes a reduction in the rate and severity of relapse.

FIG. 3 is a graphical representation of leukocyte count in bronchoalveolar lavage fluid from naive (N) or actively sensitized (AS) Brown Norway rats 72 hours following exposure to aerosolized ovalbumin (10 mg/mL). Treatment with 3b causes a dose-dependent reduction in leukocyte influx.

FIG. 4 is a graphical representation of eosinophil count in bronchoalveolar lavage fluid from naive (N) or actively sensitized (AS) Brown Norway rats 72 hours following exposure to aerosolized ovalbumin (10 mg/mL). Treatment with 3b causes a dose dependent inhibition of eosinophilia in this model.

FIG. 5 is a graphical representation of leukocyte count in bronchoalveolar lavage fluid from naive, untreated (N); actively sensitized, vehicle-treated (V); or actively sensitized, drug-treated (A-H) Brown Norway rats 72 hours following exposure to aerosolized ovalbumin (10 mg/mL). Treatment with budesonide alone (0.1 mg/kg) or 3b alone (0.03 or 0.1 mg/kg) was ineffective. However, the combination of budesonide (0.1 mg/kg) with 3b (0.03 or 0.1 mg/kg) causes a reduction in leukocyte influx in this model. High-dose budesonide (0.5 mg/kg) is efficacious with or without added 3b.

FIG. 6 is a graphical representation of cosinophil count in bronchoalveolar lavage fluid from naive, untreated (N); actively sensitized, vehicle-treated (V); or actively sensitized, drug-treated (A-H) Brown Norway rats 72 hours following exposure to aerosolized ovalbumin (10 mg/mL). Treatment with budesonide alone (0.1 mg/kg) or 3b alone (0.03 or 0.1 mg/kg) was ineffective. However, the combination of budesonide (0.1 mg/kg) with 3b (0.03 or 0.1 mg/kg) causes a reduction in eosinophilia in this model. High-dose budesonide (0.5 mg/kg) is efficacious with or without added 3b.

FIG. 7 is a graphical representation of 20 S proteasome activity in white blood cells from 7 human volunteers.

FIG. 8 is a graphical representation of daily 20 S proteasome activity in white blood cells from 7 human volunteers

FIG. 9 is a graphical representation of 20 S proteasome activity in murine white blood cells 1.0 hour after an intravenous administration of N-(pyrazine)carbonyl-L-phenylalanine-L-leucine boronic acid (1).

FIG. 10 is a graphical representation of 20 S proteasome activity in murine white blood cells 24 hours after an intravenous administration of 1.

FIG. 11 is a graphical representation of 20 S proteasome activity in rat white blood cells 1.0 hour after an intravenous administration of 1.

FIG. 12 is a graphical representation of 20 S proteasome activity in rat white blood cells 24 hours after an intravenous administration of 1.

FIG. 13 is a graphical representation of 20 S proteasome activity in rat white blood cells 48 hours after an intravenous administration of 1.

FIG. 14 is a graphical representation of 20 S proteasome activity in rat white blood cells 1.0 hour after twice weekly intravenous injections of 1 for two weeks.

FIG. 15 is a graphical representation of 20 S proteasome activity in primate white blood cells 1.0 hour after an intravenous administration of 1.

FIG. 16 is a graphical representation of 20 S proteasome activity in primate white blood cells 72 hours after an intravenous administration of 1.

FIG. 17 is a graphical representation of chymotryptic (□) and tryptic (⋄) activities as a function of the concentration of 1, demonstrating that 1 fully inhibits chymotryptic activity, but causes an activation of tryptic activity.

FIG. 18 is a graphical plot comparing percent proteasome inhibition and the ratio of chymotryptic to tryptic activities with purified 20 S proteasome from rabbit reticulocytes.

FIG. 19 is a graphical plot comparing percent proteasome inhibition and the ratio of chymotryptic to tryptic activities with rat white blood cell lysates.

### DETAILED DESCRIPTION OF THE PREFERRED EMBODIMENTS

This invention is directed to compositions and methods for treatment of inflammatory and autoimmune diseases. All patent applications, patents and literature references cited herein are hereby incorporated by reference in their entirety. In the case of inconsistencies the present disclosure will prevail.

The invention provides methods for treating a patient afflicted with multiple sclerosis or asthma comprising administering to said patient an effective amount of an agent selected from the group consisting of proteasome inhibitors, ubiquitin pathway inhibitors, agents that interfere with the activation of NF-κB via the ubiquitin proteasome pathway, and mixtures thereof. It has now been unexpectedly discovered that the ubiquitin-proteasome pathway is a target for treating multiple sclerosis, asthma, and rheumatoid arthritis.

In the present description, the following definitions will be used.

“Treating” shall mean any amelioration of any symptom pursuant to administration of any proteasome inhibitor, ubiquitin pathway inhibitor, or agent that interferes with activation of NF-κB via the ubiquitin proteasome pathway.

“Ubiquitin pathway inhibitor” shall mean any substance which specifically inhibits ubiquitination or the transfer of ubiquitin to proteins.

“Proteasome inhibitor” shall mean any substance which specifically inhibits the proteasome or the activity thereof.

“Agents that interfere with activation of NF-κB by the ubiquitin-proteasome pathway” shall mean any substance that 1) specifically inhibits the proteasome or the activity thereof; 2) specifically inhibits ubiquitination of IκB-α or p105; or 3) specifically inhibits phosphorylation of IκB-α or p105.

“Specifically inhibits” shall mean interferes with the ability of a protein to mediate its biological function at an inhibitor concentration that is lower than the concentration of the inhibitor required to produce another, unrelated biological effect. Preferably, the concentration of the inhibitor required for such interference is at least 2-fold lower, more preferably at least 5-fold lower, even more preferably at least 10-fold lower, and most preferably at least 20-fold lower than the concentration required to produce an unrelated biological effect. Such inhibitors can act by any of a variety of mechanisms, including without limitation, interfering with the active site or conformation of the protein, interfering with the ability of the protein to interact with another protein, substrate, or co-factor, either by an effect on the protein itself or on the other protein, substrate, or cofactor, and altering the microenvironment in which the biological function of the protein normally occurs.

In a first aspect, the invention provides methods for treating multiple sclerosis. Multiple sclerosis (MS) is an incurable neurological illness that frequently causes chronic disability. It is widely believed that MS is the result of an autoimmune disorder in a genetically susceptible individual, mediated by autoreactive T cells that migrate into the CNS and initiate the inflammatory demyelinating lesion. The observation that MS is an autoimmune disease is derived in part from systemic abnormalities of immune function seen in patients with the disease, and in part through similarities with experimental autoimmune encephalomyelitis (EAE), which in turn serves as a model for the human disease (Kennedy, et al. J. Neuroimmunol. (1987) 16:345; Arnason, et al. Neurol. Clin. (1983) 1:765; van der Veen, et al. J. Neuroimmunol. (1989) 48:213; Gonatas, et al. Immunol. Today (1986) 7:121; Wekerle Acta Neurol. (1991) 13:197).

EAE is a T-cell-mediated inflammatory, autoimmune demyelinating disease of the CNS. The disease can be induced in a number of experimental laboratory animals, including primates, by the injection of whole brain homogenate, a purified preparation of myelin basic protein (MBP), or proteolipoprotein (PLP) in adjuvant. EAE is a T-cell-mediated disease, and passive transfer of MBP- or PLP-reactive T cells is sufficient to induce disease. Relapsing-remitting experimental autoimmune encephalomyelitis (R-EAE) is induced in SJL/J mice by immunization with the immunodominant epitope on proteolipid protein (PLP139-151) or by the adoptive transfer of PLP139-151-specific CD4+T cells (McRae, et al. J. Neuroimmunol (1992) 38:229). The clinical disease is characterized by an acute paralytic phase followed by recovery and subsequent relapses. This pattern of relapses and spontaneous recovery in the experimental animal model, which occurs over a period of weeks to months, is very similar to the clinical signs of disease observed in multiple sclerosis (MS) patients over many years.

The method according to this aspect of the invention comprises administering to a patient afflicted with MS an effective amount of an agent selected from the group consisting of proteasome inhibitors, ubiquitin pathway inhibitors, agents that interfere with the activation of NF-κB via the ubiquitin proteasome pathway, and mixtures thereof. In a preferred embodiment, the agent is administered in an amount sufficient to reduce the frequency or severity of relapse of the disease.

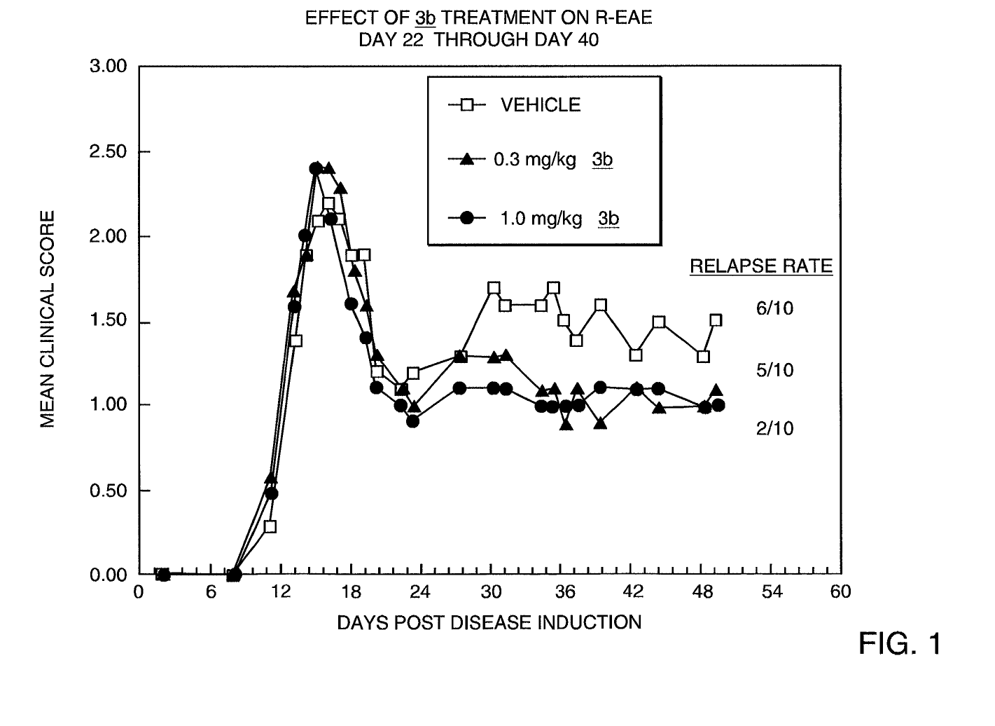

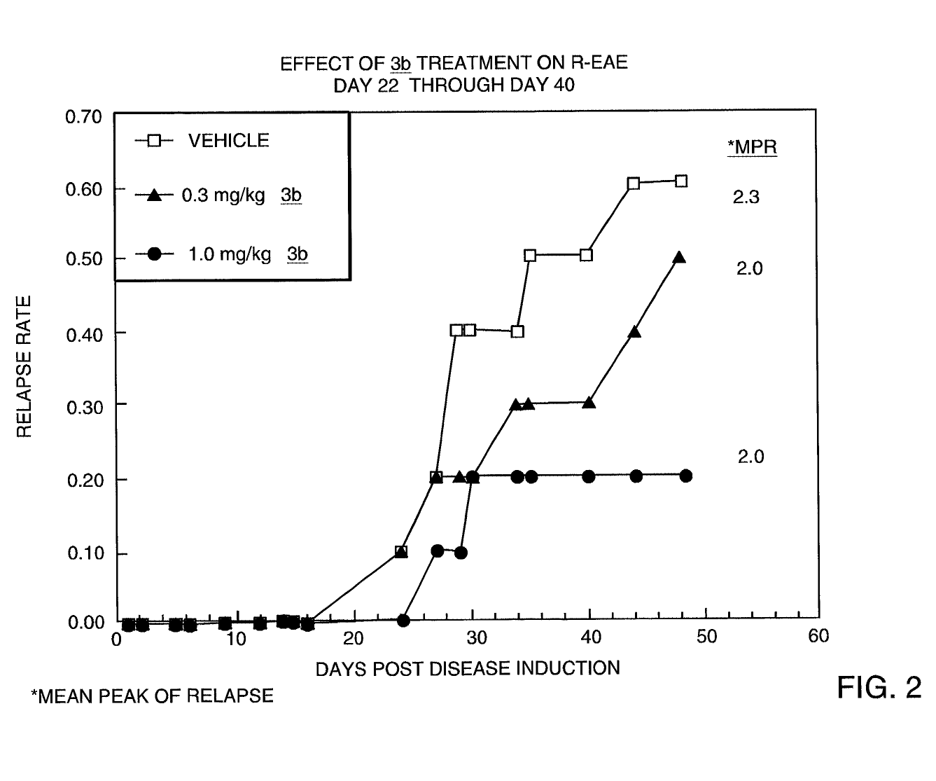

When administered during the remission phase at doses of 0.3 or 1.0 mg/kg i.p., the proteasome inhibitor 3b reduced the rate and severity of relapse in the R-EAE model (FIGS. 1-2).

In a second aspect, the invention provides a method for treating asthma. Asthma is an obstructive lung disorder characterized by airway hyperresponsiveness, which is an exaggerated airway narrowing in response to many different stimuli, such as histamine, exercise, cold air, and allergen. Because of the episodic constriction of the bronchial tubes, treatment has been based partly on bronchodilation by β-adrenergic agonist drugs. More recently, however, it has become appreciated that asthma is a chronic condition of the airways that involves many types of inflammatory cell and the release of many mediators and neurotransmitters that have multiple effects on the various target cells in the airway. The degree and extent of inflammation in the airway wall are broadly related to the clinical severity of the asthma. The inflammatory response of asthma consists of activation of mast cells resident in the airways, increased numbers of lymphocytes (which are mainly CD4+T lymphocytes) and an infiltration with eosinophils, which appear to degranulate. Increased total eosinophil count in the peripheral blood is almost invariably present unless suppressed by corticosteroids or sympathomimetic drugs. Sputum examination also reveals eosinophils.

Several animal models have been developed to study pulmonary inflammation with characteristic manifestations of airways eosinophilia. One of the often-used animal models is the ovalbumin sensitized guinea pig (Dunn, et al. Am. Rev. Respir. Dis. (1990) 142:680; Sanjar, et al. Br. J. Pharmacol. (1990) 99:679; Gulbenkian, et al. Am. Rev. Respir. Dis. (1990) 142:680). Selective accumulation of both neutrophils and eosinophils have also been described in acutely sensitized Brown Norway rats (Kips, et al. Am Rev. Respir. Dis. (1992) 145:1306; Richards, et al. Agents Actions, Suppl. 34 (1991) 34:359; Chapman, et al. Am. J. Resp. Crit. Care Med. (1996) 153:A219). The allergen-induced pulmonary eosinophilia in actively sensitized Brown Norway rats is inhibited by the steroid dexamethasone. Glucocorticoid therapy remains one of the most effective anti-inflammatory treatments available, and these drugs have been shown to reduce pulmonary eosinophilia in asthmatic patients (Holgate, et al. Int. Arch. Allergy Appl. Immunol. (1991) 94:210).

The method according to this aspect of the invention comprises administering to a patient afflicted with asthma an effective amount of an agent selected from the group consisting of proteasome inhibitors, ubiquitin pathway inhibitors, agents that interfere with the activation of NF-κB via the ubiquitin proteasome pathway, and mixtures thereof. In a preferred embodiment, the agent is administered in an amount sufficient to reduce the frequency or severity of asthmatic attacks.

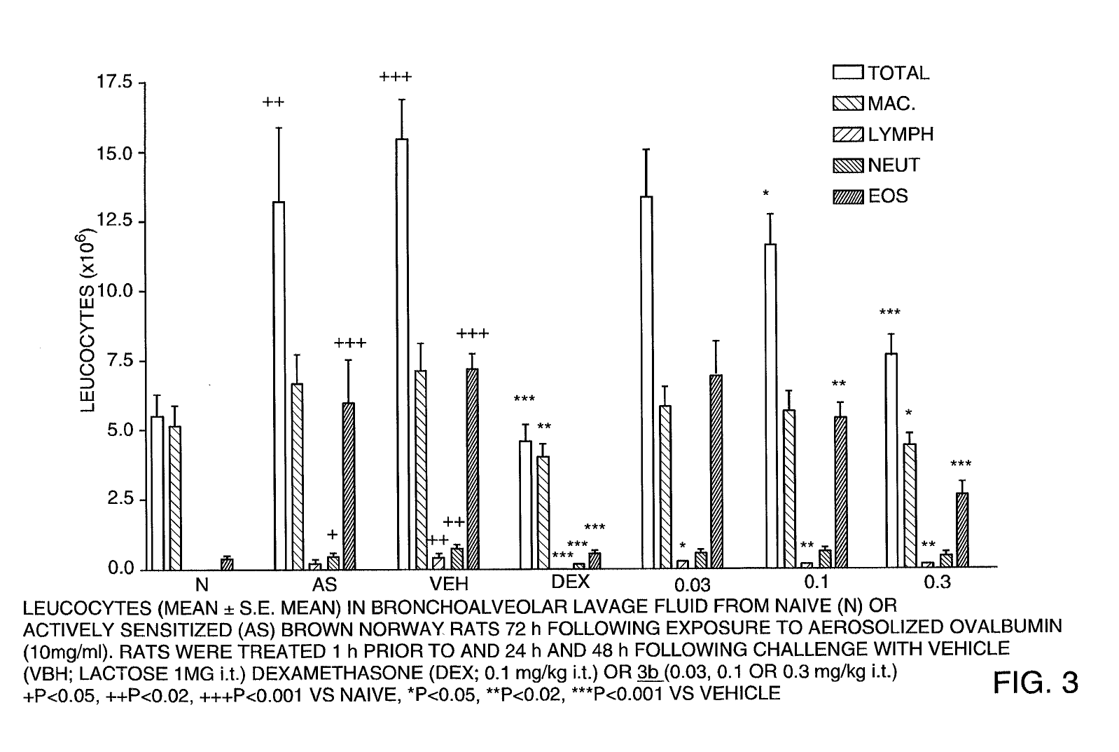

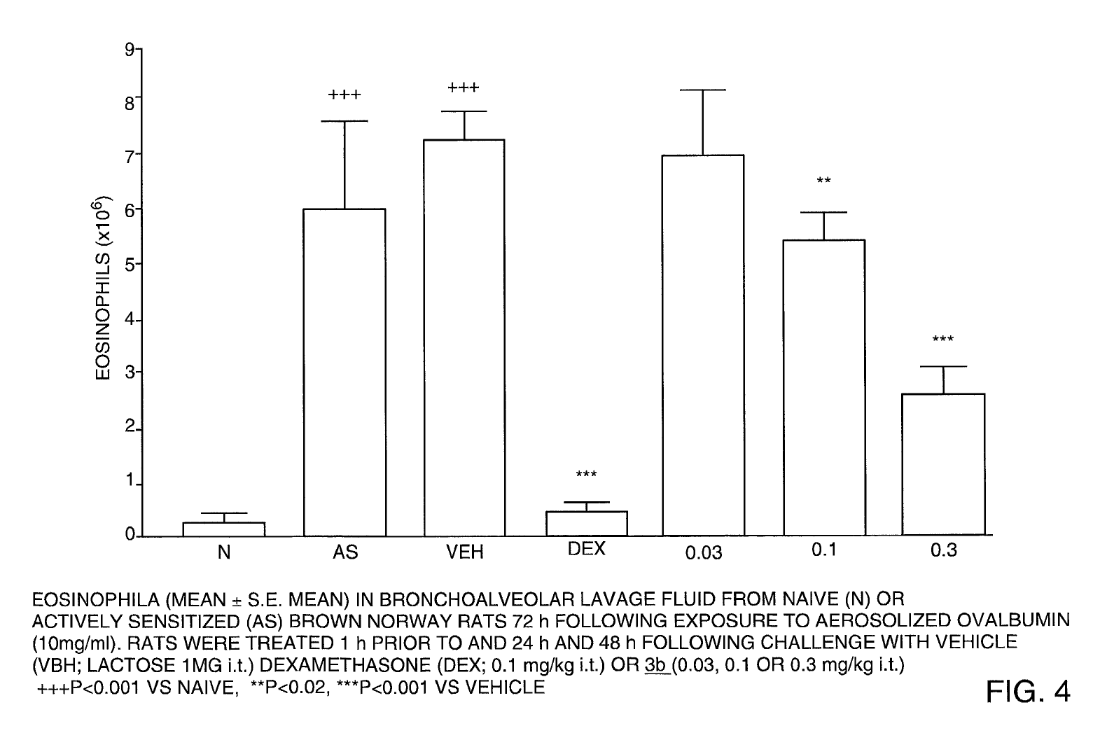

When administered intratracheally at 1 hour prior to and 24 hours and 48 hours after allergen challenge, 3b (0.1 or 0.3 mg/kg) inhibited eosinophilia in actively sensitized Brown Norway rats (FIGS. 3-4).

Further contemplated within the scope of the invention is combined administration with another drug or drugs used to treat asthma. Currently accepted therapies for asthma include cromoglycate, nedocromil, theophylline, short- and long-acting β₂-adrenergic receptor agonists, and inhaled or oral glucocorticoids. More recently developed therapeutics include inhibitors of leukotriene biosynthesis, leukotriene receptor antagonists, and thromboxane antagonists. Anti-IL-5 and anti-IgE antibodies are being developed (Science (1997) 276:1643), and antisense approaches are also being investigated (Nyce and Metzger, Nature (1997) 383:721). In one preferred embodiment, the agent of the invention is used in an amount sufficient to reduce the dose or treatment frequency required for the other drug or drugs. In another preferred embodiment, the other drug or drugs are used in an amount sufficient to reduce the dose or treatment frequency required for the agent of the invention. The agent may be administered at the same time as the other drug or drugs or may be administered at a different time.

Steroid therapy is particularly effective for the treatment of asthma, and is an essential line of therapy for severe asthmatics. Unfortunately, however, a number of untoward side-effects result from long-term steroid use, including bone growth suppression, adrenal insufficiency, Cushing's syndrome, cataracts, immunosuppression, and excessive bruising. Many of these effects can be minimized by topical administration of the drug to the lung by inhalation. However, high doses, such as those required in severe cases, result in significant systemic exposure and an increase in the associated side-effects. Drugs that permit the reduction in steroid dose (“steroid-sparing”) thus offer very real clinical benefit.

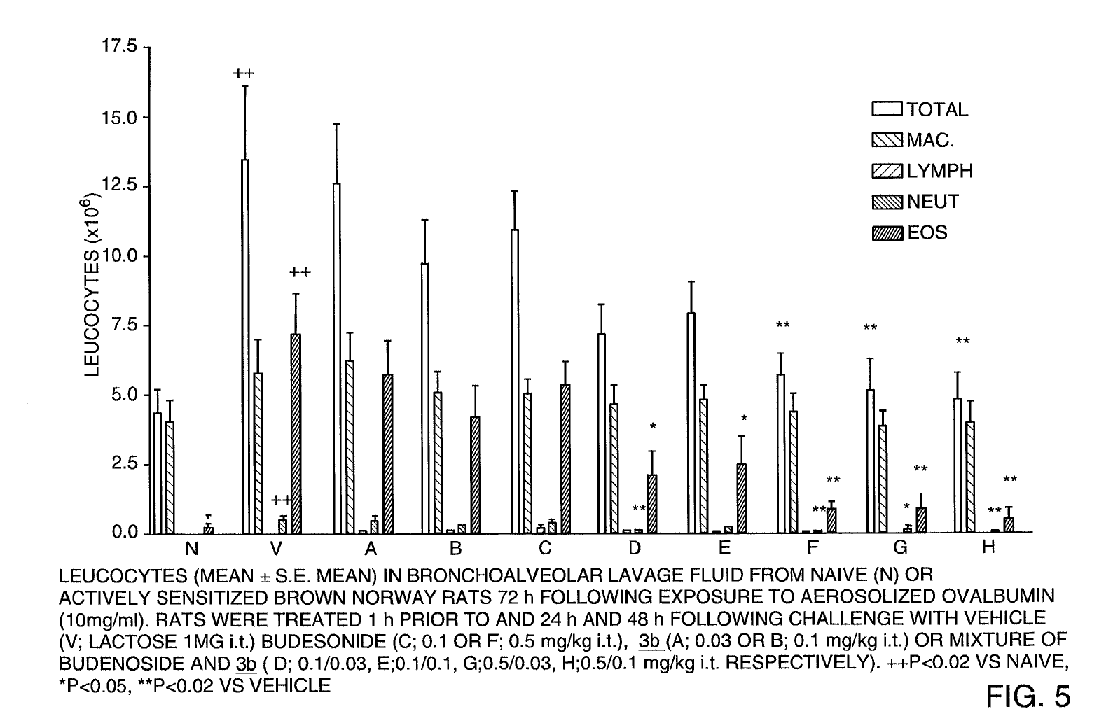

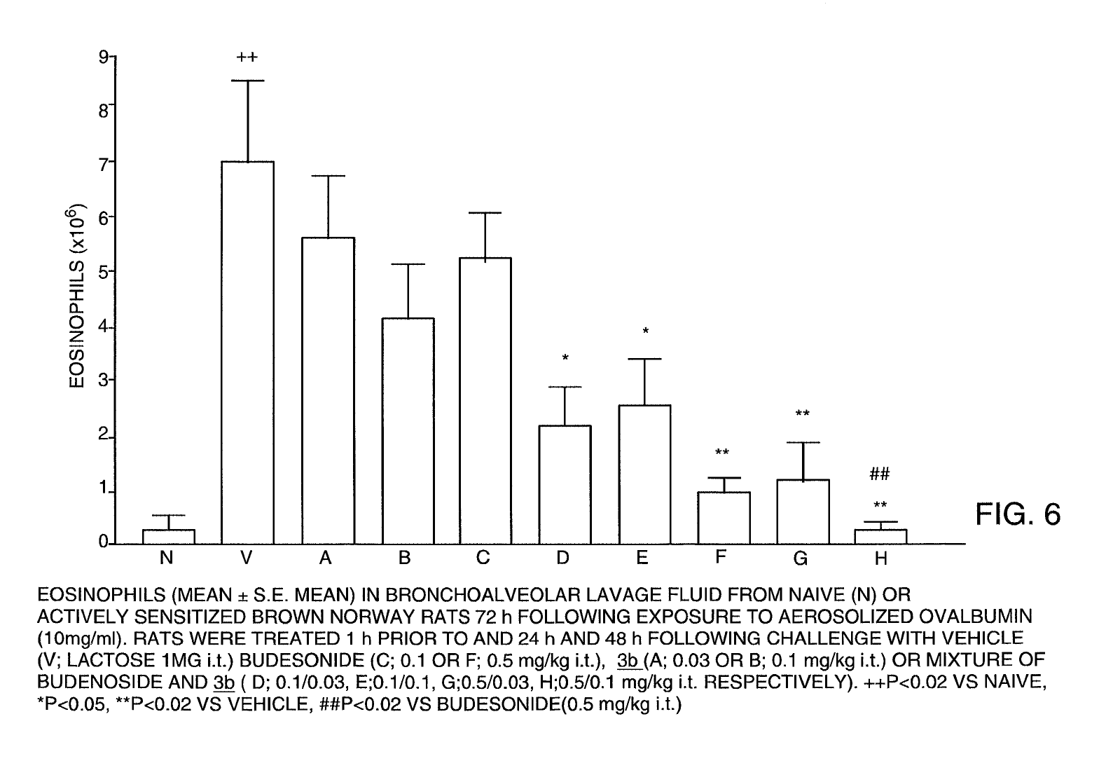

Unexpectedly, it has been found that intratracheal administration of 3b (0.03 or 0.1 mg/kg) in combination with the glucocorticoid budesonide (0.1 mg/kg) at 1 hour prior to and 24 hours and 48 hours after allergen challenge inhibits eosinophilia in actively sensitized Brown Norway rats (FIGS. 5-6). Strikingly, neither drug was effective when administered alone at these doses, suggesting synergistic action of the two drugs.

In a third aspect, the invention provides methods for treating a patient afflicted with asthma comprising administering to the patient a combination of a glucocorticoid and an agent selected from the group consisting of proteasome inhibitors, ubiquitin pathway inhibitors, agents that interfere with the activation of NF-κB via the ubiquitin proteasome pathway, and mixtures thereof. The glucocorticoid and the agent may be administered at the same or different times, on the same or different days, and with the same or different frequency. Preferably, the doses of each drug are spaced so as to achieve a combined physiological effect. Preferably, the glucocorticoid is administered between 0 minutes and about one month before or after the agent of the invention, more preferably between 0 minutes and about one week before or after the agent of the invention, mo st preferably between 0 minutes and 24 hours before or after the agent of the invention.

Glucocorticoids for use in the invention include, but are not limited to, flunisolide, triamcinolone acetonide, beclomethasone dipropionate, dexamethasone sodium phosphate, fluticasone propionate, budesonide, hydrocortisone, prednisone, prednisolone, mometasone, tipredane, and butixicort. Preferably, the glucocorticoid is budesonide. Suitable formulations, dosages, and routes of administration for glucocorticoids are known in the art (Physician's Desk Reference, 51st Edition, 1997, Medical Economics: Montvale, N.J.).

In certain preferred embodiments, the agent of the invention is administered in an amount sufficient to reduce the dose or treatment frequency required for the glucocorticoid. Preferably, the amount of glucocorticoid administered does not exceed the standard recommended dosage, and more preferably the amount of glucocorticoid administered is less than the standard recommended dosage for the drug when administered alone.

In certain preferred embodiments, the amount of glucocorticoid administered is sufficient to reduce the dose or treatment frequency required for the agent selected from the group consisting of proteasome inhibitors, ubiquitin pathway inhibitors, agents that interfere with the activation of NF-κB via the ubiquitin proteasome pathway, and mixtures thereof. Most preferably, treatment of a patient afflicted with asthma with a combination of a glucococorticoid and an agent selected from the group consisting of proteasome inhibitors, ubiquitin pathway inhibitors, agents that interfere with the activation of NF-κB via the ubiquitin proteasome pathway, and mixtures thereof produces efficacy with fewer or less severe side effects or toxicity than treatment with either drug alone.

In a fourth aspect, the invention provides pharmaceutical compositions comprising a combination of a glucocorticoid and an agent selected from the group consisting of proteasome inhibitors, ubiquitin pathway inhibitors, agents that interfere with the activation of NF-κB via the ubiquitin proteasome pathway, and mixtures thereof, are further contemplated within the scope of the invention. The pharmaceutical compositions of the invention can be provided in unit dosage form. In a preferred embodiment, the unit dosage form contains an amount of glucocorticoid that is less than its standard recommended dosage when administered by itself. The following description of non-limiting examples of suitable proteasome inhibitors, ubiquitin pathway inhibitors, and agents that interfere with the activation of NF-κB via the ubiquitin proteasome pathway, applies to the pharmaceutical formulations as well as to the methods according to the invention.

Non-limiting examples of proteasome inhibitors for use in the present invention include peptidyl aldehydes (Orlowski et al. U.S. Pat. No. 5,580,854; Stein et al. WO 95/24914; Siman et al. WO 91/13904; Iqbal et al. J. Med. Chem. 38:2276-2277 (1995)), peptidyl boronic acids (Adams et al. WO 96/13266; Siman et al. WO 91/13904), other peptidyl derivatives with proteasome inhibitory activity (Iqbal et al. U.S. Pat. No. 5,614,649; Iqbal et al. U.S. Pat. No. 5,550,262; Spaltenstein et al. Tetrahedron Letters 1996, 37, 1343), and lactacystin and lactacystin analogs (Fenteany et al. Proc. Natl. Acad. Sci. USA (1994) 91:3358; Fenteany et al. WO 96/32105; Soucy et al. U.S. patent application Ser. No. 08/912,111, filed Aug. 15, 1997)). The agents disclosed herein may be administered by any route, including intradermally, intraperitoneally, intranasally, intratracheally, subcutaneously, orally or intravenously. For asthma indications, administration is preferably by the inhalation route.

Peptide aldehyde proteasome inhibitors for use in the present invention preferably are those disclosed in Stein et al. WO 95/24914 published Sep. 21, 1995 or Siman et al. WO 91/13904 published Sep. 19, 1991, both hereby incorporated by reference in their entirety.

Boronic acid or ester compounds for use in the present invention preferably include those disclosed in Adams et al. WO 96/13266, Siman et al. WO 91/13904, or Iqbal et al. U.S. Pat. No. 5,614,649, each of which is hereby incorporated by reference in its entirety.

In certain preferred embodiments, the boronic acid compound for use in the present invention is selected from the group consisting of:

N-acetyl-L-leucine-β-(1-naphthyl) L-alanine-L-leucine boronic acid,

β-(1-naphthyl)-L-alanine-L-leucine boronic acid,

N-(4-morpholine)carbonyl-β-(1 -naphthyl)-L-alanine-L-leucine boronic acid,

N-(8-quinoline)sulfonyl-β-(1-naphthyl)-L-alanine-L-leucine boronic acid,

N-(2-pyrazine)carbonyl-L-phenylalanine-L-leucine boronic acid, and

N-(4-morpholine)carbonyl-[O-(2-pyridylmethyl)]-L-tyrosine-L-leucine boronic acid.

Lactacystin and lactacystin analog compounds for use in the present invention preferably include those disclosed in Fenteany et al. WO 96/32105, or Soucy et al. U.S. patent application Ser. No. (08/912,111; filed Aug. 15, 1997), each of which is hereby incorporated by reference in its entirety. In certain preferred embodiments, the lactacystin analog compound is selected from the group consisting of lactacystin, clasto-lactacystin β-lactone, 7-ethyl-clasto-lactacystin β-lactone, 7-n-propyl-clasto-lactacystin β-lactone, and 7-n-butyl-clasto-lactacystin β-lactone. These compounds can be prepared as illustrated in Schemes 1 and 2. Most preferably, the lactacystin analog compound is 7-n-propyl-clasto-lactacystin β-lactone (3b (Scheme 2)).

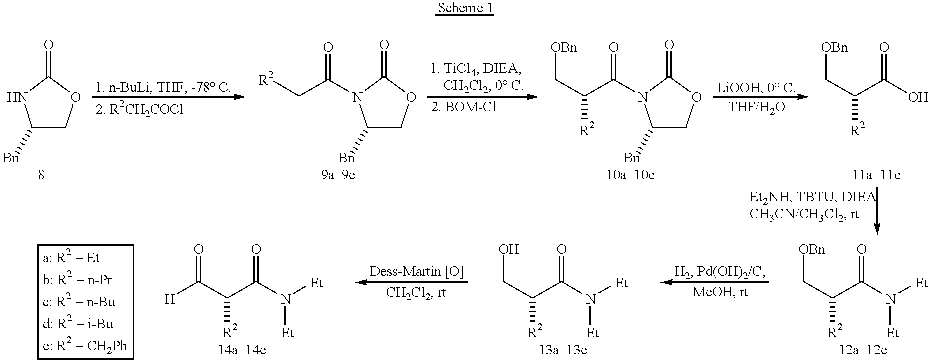

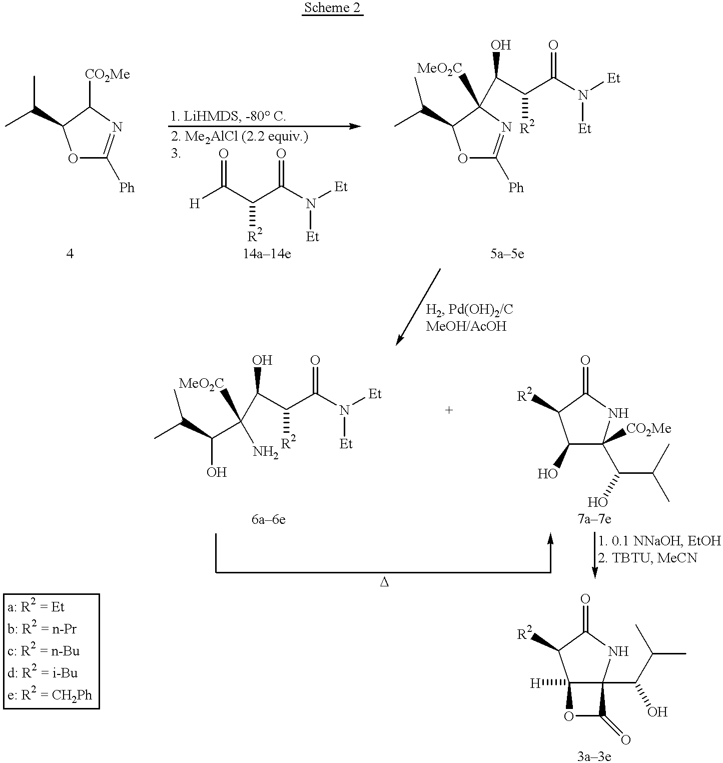

In a preferred embodiment, the agent used to treat a patient afflicted with multiple sclerosis or asthma is a proteasome inhibitor. Preferably the proteasome inhibitor is lactacystin or a lactacystin analog, more preferably 7-n-propyl-clasto-lactacystin β-lactone. Preferably, the combination used to treat a patient afflicted with asthma comprises a glucocorticoid and a proteasome inhibitor. More preferably, the proteasome inhibitor is lactacystin or a lactacystin analog. Most preferably the combination comprises 7-n-propyl-clasto-lactacystin β-lactone and budesonide.

Non-limiting examples of ubiquitin pathway inhibitors include those disclosed in Berleth et al, Biochem. 35(5): 1664-1671, (1996). Inhibitors of IκB-α phosphorylation are also known (Chen, Cell 84:853 (1996); Chen U.S. patent application Ser. No. 08/825,559).

The concentration of a disclosed compound in a pharmaceutically acceptable mixture will vary depending on several factors, including the dosage of the compound to be administered, the pharmacokinetic characteristics of the compound(s) employed, and the route of administration. Effective amounts of agents for treating multiple sclerosis, asthma, or rheumatoid arthritis would broadly range between about 10 μg and about 50 mg per Kg of body weight of a recipient mammal. The agent may be administered in a single dose or in repeat doses. Treatments may be administered daily or more frequently depending upon a number of factors, including the age and overall health of a patient, and the formulation and route of administration of the selected compound(s). Other factors to be considered in determining dosage include kind of concurrent treatment, if any; frequency of treatment and the nature of the effect desired; extent of tissue damage; gender; duration of symptoms; counter indications, if any; and other variables to be assessed by the individual physician.

In certain preferred embodiments, the dose regimen for the proteasome inhibitor is determined by measuring the activity of the proteasome activity ex vivo after administering the proteasome inhibitor to the mammal, as described in U.S. application Ser. No. 60/131,381, filed Apr. 28, 1999, the entire contents of which are hereby expressly incorporated by reference. Such measurement comprises obtaining one or more test biological samples from the mammal at one or more specified times after administering the proteasome inhibitor; measuring proteasome activity in the test biological sample or samples; determining the amount of proteasome activity in the test biological sample or samples; comparing the amount of proteasome activity in the test biological sample to that in a reference biological sample obtained from a mammal to which no proteasome inhibitor has been administered; and selecting a dose amount and dose frequency of the proteasome inhibitor to be administered in the future.

The biological samples that are obtained from the mammal may include, without limitation, blood, urine, organ, and tissue samples. In certain preferred embodiments, the biological sample is obtained from a locus of disease. In one preferred embodiment, the biological sample comprises bronchial fluid from a patient with asthma.

In certain other preferred embodiments, the biological sample is a blood sample, more preferably a blood cell lysate. Cell lysis may be accomplished by standard procedures. In certain preferred embodiments, the biological sample is a whole blood cell lysate. Kahn et al. (Biochem. Biophys. Res. Commun., 214:957-962 (1995)) and Tsubuki et al. (FEBS Lett., 344:229-233 (1994)) disclose that red blood cells contain endogenous proteinaceous inhibitors of the proteasome. Thus, contamination of biological samples with even small amounts of red blood cells could interfere with the assay. However, endogenous proteasome inhibitors are inactivated in the presence of SDS at a concentration of about 0.05%, allowing red blood cell lysates and whole blood cell lysates to be assayed reliably. At this concentration of SDS, all proteasome activity is due to the 20 S proteasome. Although purified 20 S proteasome exhibits poor stability at 0.05% SDS, 20 S proteasome activity in cell lysates is stable under these conditions. The ability to perform the assay in whole blood cell lysates offers significant advantages in terms of economy and ease of sample preparation.

In certain other preferred embodiments, the biological sample is a white blood cell lysate. Methods for fractionating blood cells are known in the art (Rickwood et al., Anal. Biochem. 123:23-31 (1982); Fotino et al., Ann. Clin. Lab. Sci. 1:131 (1971)) and are further described in the Examples. Commercial products useful for cell separation include without limitation FICOLL-PAQUE^{™} (Pharmacia Biotech) and NYCOPREP^{™} (Nycomed). In some situations, white blood cell lysates provide better reproducibility of data than do whole blood cell lysates and, therefore, may be preferred in those situations.

Variability in sample preparation can be corrected for by introducing a normalization step into the workup of the data. In certain preferred embodiments, proteasome activity in the sample may be normalized relative to the protein content in the sample (specific activity method). Total protein content in the sample can be determined using standard procedures, including, without limitation, Bradford assay and the Lowry method. In certain other preferred embodiments, proteasome activity in the sample may be normalized relative to cell count. This embodiment may be preferred in settings, such as clinical settings, in which automated cell counters are readily accessible.

Proteasome inhibitors often exhibit preferential inhibition of one peptidase activity of the proteasome over other proteasome peptidase activities. The present inventors have recognized that this differential inhibition provides an alternative approach to normalization procedures based on protein content or cell count. Thus, in certain particularly preferred embodiments, proteasome inhibition is determined as a ratio of one peptidase activity of the proteasome to another. The derivation of the theoretical equation for determination of proteasome inhibition according to this embodiment of the invention is provided in the Examples. In order for this embodiment of the invention to be operative, the proteasome inhibitor under study must inhibit one peptidase activity preferentially over at least one other peptidase activity. Selection of the peptidase activities to be assayed, and thus the appropriate peptidic substrates to be used, will depend on the inhibitor under study. For example, for the inhibitor N-(pyrazine)carbonyl-L-phenylalanine-L-leucine boronic acid (1), proteasome inhibition is preferably determined as a ratio of chymotryptic activity to tryptic activity. Chymotryptic activity is fully inhibitable by 1, whereas tryptic activity is activated by 1 over the same concentration range.

Proteasome activity in the biological sample is measured by any assay method suitable for determining 20 S or 26 S proteasome activity. (See, e.g., McCormack et al., Biochemistry 37:7792-7800 (1998)); Driscoll and Goldberg, J. Biol. Chem. 265:4789 (1990); Orlowski et al., Biochemistry 32:1563 (1993)). Preferably, a substrate having a detectable label is provided to the reaction mixture and proteolytic cleavage of the substrate is monitored by following disappearance of the substrate or appearance of a cleavage product. Detection of the label may be achieved, for example, by fluorometric, colorimetric, or radiometric assay.

Preferred substrates for determining 26 S proteasome activity include, without limitation, lysozyme, α-lactalbumin, b-lactoglobulin, insulin b-chain, and ornithine decarboxylase. When 26 S proteasome activity is to be measured, the substrate is preferably ubiquitinated or the reaction mixture preferably further comprises ubiquitin and ubiquitination enzymes.

More preferably, the substrate is a peptide less than 10 amino acids in length. In one preferred embodiment, the peptide substrate contains a cleavable fluorescent label and release of the label is monitored by fluorometric assay. Non-limiting examples of preferred substrates according to this embodiment of the invention include N-(N-carbobenzyloxy-carbonylleucylleucylarginyl)-7-amino-4-methylcoumarin (Z-Leu-Leu-Arg-AMC), N-(N-benzoylvalylglycylarginyl)-7-amino-4-methylcoumarin (Bz-Val-Gly-Arg-AMC), N-(N-carbobenzyloxycarbonylleucylleucylarginyl)-2-naphthylamine (Z-Leu-Leu-Glu-2NA), or N-(N-succinylleucylleucylvalyltyrosyl)-7-amino-4-methylcoumarin (Suc-Leu-Leu-Val-Tyr-AMC). In certain preferred embodiments, the reaction mixture further comprises a 20 S proteasome activator. Preferred activators include those taught in Coux et al. (Ann. Rev. Biochem. 65: 801-847 (1995)), preferably PA28 or sodium dodecyl sulfate (SDS).

Day-to-day variability in the assay may result from factors such as differences in buffer solutions, operator variability, variability in instrument performance, and temperature variability. Such variability can be minimized by standardizing proteasome activity in both the biological sample and the reference sample relative to a standard proteasome sample comprising a known or constant amount of proteasome activity. In certain preferred embodiments, the standard sample comprises purified 20 S proteasome, more preferably purified 20 S proteasome from a eukaryote. The source of 20 S proteasome is not critical and includes without limitation mammals, including without limitation rabbits. In certain preferred embodiments, the 20 S proteasome is purified from rabbit reticulocytes. In certain other preferred embodiments, the standard sample is a biological sample, including, without limitation, a blood sample. Preferably, the biological sample is a whole blood cell lysate, more preferably a whole blood cell lysate obtained from a human, preferably a human who has not been exposed to proteasome inhibitor administration.

The proteasome activity measured in the test biological sample is compared to that measured in a reference biological sample obtained from a mammal to which no proteasome inhibitor has been administered. In some preferred embodiments, the test biological sample and the reference biological sample each separately comprise a plurality of samples pooled from a group of mammals, preferably mice, undergoing treatment. In other preferred embodiments, the test biological sample and the reference biological sample each comprise a single sample obtained from an individual mammal. Assaying of individual samples is presently preferred except when impractical due to the small size of the mammal. In some preferred embodiments, a statistical sample is obtained by pooling data from individual test biological samples or from individual reference biological samples.

In some preferred embodiments, the reference sample is obtained from the treated mammal prior to initiation of proteasome inhibitor treatment. This embodiment is presently preferred for higher mammals in order to minimize the impact of inter-mammal variability. Clinical monitoring of proteasome inhibitor drug action presently preferably entails this embodiment of the invention, with each patient serving as his or her own baseline control.

A decrease in proteasome activity in the biological sample as compared to the reference sample is indicative of an in vivo effect of the proteasome inhibitor at the time the biological sample was obtained. In some preferred embodiments, biological samples are obtained at multiple time points following administration of the proteasome inhibitor. In these embodiments, measurement of proteasome activity in the biological samples provides an indication of the extent and duration of in vivo effect of the proteasome inhibitor. In certain other preferred embodiments, multiple biological samples are obtained from a single mammal at one or more time points. In these embodiments, measurement of proteasome activity in the biological samples provides an indication of the distribution of the proteasome inhibitor in the mammal.

Potential sources of variability in proteasome activity measurements include inter-individual differences, fluctuations in proteasome activity in a single individual over time, and differences in proteasome activity in white blood cells and red blood cells. All three sources of variability may impact proteasome inhibition determinations based on specific activity. By contrast, proteasome inhibition determinations based on the ratio of one peptidase activity of the proteasome to another may exhibit greater consistency.

Dose amount may preferably be determined on a mg/kg or mg/m² basis. The mammal to which the future dose is to be administered may be the same mammal as that from which the biological sample or samples were obtained, or it may be a different mammal. In some embodiments, the above recited steps may be repeated. For example, in a clinical setting, the dose amount and dose frequency may be repeatedly or continuously adjusted as a result of repeated monitoring of proteasome activity in biological samples obtained from the patient.

In certain preferred embodiments, the dose amount and dose frequency of the proteasome inhibitor are selected so as to avoid excessive proteasome inhibition. In some embodiments, excessive proteasome inhibition results in a toxic effect, the toxic effect including, but not being limited to, vomiting, diarrhea, hypovolemia, hypotension, and lethality. Preferably the dose amount and dose frequency of the proteasome inhibitor are selected so that proteasome inhibition in any future biological sample does not exceed about 95%.

In certain other preferred embodiments, the dose amount and dose frequency of the proteasome inhibitor are selected so that therapeutically useful proteasome inhibition is achieved. Preferably, therapeutically useful proteasome inhibition results in a therapeutically beneficial antiinflammatory or immunosuppressive effect. Preferably, the dose amount and dose frequency of the proteasome inhibitor are selected so that proteasome inhibition of at least about 15%, preferably about 20%, more preferably about 30%, even more preferably about 40%, and still more preferably about 50%, and most preferably from about 50% to about 80% is achieved in a future biological sample, although in some instances proteasome inhibition as high as 95% may be preferred. Agents for use in this invention may be prepared for administration by any of the methods well known in the pharmaceutical art, for example, as described in Remington's Phannaceutical Sciences (Mack Pub. Co., Easton, Pa., 1980). Agents may be prepared for use in parenteral administration in the form of solutions or liquid suspensions; for oral administration in the form of tablets or capsules; for intranasal or intratracheal administration in the form of powders, gels, oily solutions, nasal drops, aerosols, or mists. Formulations for parenteral administration may contain as common excipients sterile water or sterile saline, polyalkylene glycols such as polyethylene glycol, oils of vegetable origin, hydrogenated naphthalenes, and the like. Controlled release of an agent may be obtained, in part, by use of biocompatible, biodegradable polymers of lactide, and copolymers of lactide/glycolide or polyoxyethylene/polyoxypropylene. Additional parenteral delivery systems include ethylene-vinyl acetate copolymer particles, osmotic pumps, implantable infusion systems, and liposomes. Formulations for inhalation administration may contain lactose, polyoxyethylene-9-lauryl ether, glycocholate, or deoxycholate.

For the treatment of asthma, the inhalation route of administration is preferred in order to minimize potential side effects or toxicity resulting from systemic exposure to the agent.

According to the present invention, an “effective amount” an agent is an amount sufficient to produce any amelioration of any symptom or sign of the disease (Stites et aL Basic & Clinical Immunology Lange Medical Publications, Los Altos, Calif., 1982).

The use of any of the agents disclosed herein in combination with another agent or agents used in the treatment of multiple sclerosis or asthma is further contemplated within the scope of the present invention.

The invention is further exemplified by the following non-limiting examples:

### EXAMPLES

### Example 1

### Relapsing-Remitting Experimental Autoimmune Encephalomyelitis

Materials and Methods

Mice. Female SJL/J mice, 6 weeks old, were purchased from Harlan Laboratories (Indianapolis, Ind.), housed in the Northwestern animal care facility, and maintained on standard laboratory food and water ad libitum. Paralyzed mice were afforded easier access to food and water.

Peptides. PLP139-151 (HSLGKWLGHPDKF) was purchased from Peptides International (Louisville, Ky.). Amino acid composition was verified by mass spectrometry and purity (>98%) was assessed by HPLC.

Induction of R-EAE. Mice were immunized by subcutaneous injection of PLP139-151 in complete Freund's adjuvant (CFA) as previously described (McRae, et al. J. Neuroimmunol. (1992) 38:229). Each mouse received 50 μg of PLP139-151 distributed over 2 sites on each hind flank.

Drug Treatment. Starting on day 22, animals (10 per group) were dosed once daily i.p. (5 mL/kg) with vehicle or with 3b (0.3 or 1.0 mg/kg). Treatment continued through day 40.

Clinical Evaluation. Mice were observed daily for clinical signs of disease. Mice were scored according to their clinical severity as follows: grade 0, no abnormality; grade 1, limp tail; grade 2, limp tail and hind limb weakness (waddling gait); grade 3, partial hind limb paralysis; grade 4, complete hind limb paralysis; and grade 5, moribund.

Results

Data are plotted as the mean daily clinical score for all animals in a particular treatment group (FIG. 1). A relapse was defined as an increase of at least one full grade in clinical score after the animal had previously improved at least a full clinical score and had stabilized. Animals treated with 3b (both dosage groups) showed reduced clinical scores as compared to vehicle-treated animals. The incidence of relapse was 5/10 for the 0.3 mg/kg group and 2/10 for the 1.0 mg/kg group, as compared to 6/10 for the vehicle-treated group.

Data are also plotted as mean daily relapse incidence for all animals in a particular treatment group (FIG. 2). The mean maximal clinical score per group is also provided as an indication of disease severity. Animals treated with 3b (both dosage groups) showed reduced rate of relapse and reduced severity of disease as compared with vehicle-treated animals.

### Example 2

### Effect of Treatment With 3b on Allergen-Induced Pulmonary Leukocyte Accumulation in Actively Sensitized Brown Norway Rats

Materials and Methods

Rats. Male Brown Norway rats were supplied by Harlan Olac Limited (Bicester, Oxon, UK) and delivered within the weight range of 180-200 g. Following acclimatization for at least five days, animals were actively sensitized over a 3-week period and were within the weight range 250-300 g at the time of allergen exposure. Food and water were provided ad libitum.

Sensitization. Ovalbumin (OA; 10 μg) mixed with aluminum hydroxide gel (10 mg) will be injected (0.5 mL, i.p.) into Brown Norway rats and repeated 7 and 14 days later.

Drug Treatment. On day 21, sensitized rats were anaesthetized (halothane 5% in O₂) and 3b, dexamethasone, or vehicle (lactose) was instilled via a cannula placed directly into the trachea at 1 hour prior to OA exposure. This procedure was repeated at 24 hours and 48 hours after OA exposure.

Challenge. Following recovery, sensitized animals were restrained in plastic tubes and exposed (60 min) to an aerosol of OA (10 mg/mL) using a nose-only exposure system. Animals were sacrificed 72 hours later with pentobarbital (250 mg/kg i.p.).

Analysis. The lungs were lavaged using 3 aliquots (4 mL) of Hank's solution (HBSS x 10, 100 mL; EDTA 100 mM, 100 mL; HEPES 1M, 10 mL made to 11 mL with water); recovered cells were pooled and the total volume of recovered fluid was adjusted to 12 mL by addition of Hank's solution. Total cells were counted (Sysmex Microcell Counter F-500, TOPA Medical Electronics Ltd., Japan). Smears were made by diluting recovered fluid (to approximately 10⁶ cells/mL) and spinning an aliquot (100 μL) in a centrifuge (Cytospin, Shandon, UK). Smears were air dried, fixed using a solution of fast green in methanol (2 mg/L) for 5 seconds and stained with eosin G (5 seconds) and thiazine (5 seconds) (Diff-Quik, Baxter Dade Ltd, Switzerland) in order to differentiate eosinophils, neutrophils, macrophages and lymphocytes. A total of 500 cells per smear were counted by light microscopy under oil immersion (x 1000).

Results

Ovalbumin challenge resulted in a significant increase in eosinophils, neutrophils, and total leukocytes in BAS fluid from actively sensitized (AS) Brown Norway rats as compared to naive (N) rats. Treatment with dexamethasone (0.1 mg/kg i.t.) prevented this increase. At doses of 0.1 and 0.3 mg/kg, 3b reduced the influx of eosinophils and total leukocytes. A significant decrease in lymphocyte count was also observed at all doses (FIGS. 3-4).

Conclusion

Compound 3b is effective in preventing leukocyte influx following allergen challenge in an animal model of asthma.

### Example 3

### Effect of Treatment with a Combination of 3b and Budesonide on Allergen-Induced Pulmonary Leukocyte Accumulation in Actively Sensitized Brown Norway Rats

Materials and Methods

Rats. Male Brown Norway rats were supplied by Harlan Olac Limited (Bicester, Oxon, UK) and delivered within the weight range of 180-200 g. Following acclimatization for at least five days, animals were actively sensitized over a 3-week period and were within the weight range 250-300 g at the time of allergen exposure. Food and water were provided ad libitum.

Sensitization. Ovalbumin (OA; 10 μg) mixed with aluminum hydroxide gel (10 mg) will be injected (0.5 mL, i.p.) into Brown Norway rats and repeated 7 and 14 days later.

Drug Treatment. On day 21, sensitized rats were anaesthetized (halothane 5% in O₂) and treated intratracheally (i.t.) 1 hour prior to OA exposure with vehicle (group V; lactose, 1 mg), budesonide (group C, 0.1 mg/kg; group F, 0.5 mg/kg), 3b (group A, 0.03 mg/kg; group B, 0.1 mg/kg), or mixtures of budesonide and 3b (group D, 0.1/0.03 mg/kg; group E, 0.1/0.1 mg/kg; group G, 0.5/0.03 mg/kg; group H, 0.5/0.1 mg/kg). Drug was instilled via a cannula placed directly into the trachea. This procedure was repeated at 24 hours and 48 hours after OA exposure.

Challenge. Following recovery, sensitized animals were restrained in plastic tubes and exposed (60 min) to an aerosol of OA (10 mg/mL) using a nose-only exposure system. Animals were sacrificed 72 hours later with pentobarbital (250 mg/kg i.p.).

Analysis. The lungs were lavaged using 3 aliquots (4 mL) of Hank's solution (HBSS x 10,100 mL; EDTA 100 mM, 100 mL; HEPES 1M, 10 mL made to 11 mL with water); recovered cells were pooled and the total volume of recovered fluid was adjusted to 12 mL by addition of Hank's solution. Total cells were counted (Sysmex Microcell Counter F-500, TOPA Medical Electronics Ltd., Japan). Smears were made by diluting recovered fluid (to approximately 10⁶ cells/mL) and spinning an aliquot (100 μL) in a centrifuge (Cytospin, Shandon, UK). Smears were air dried, fixed using a solution of fast green in methanol (2 mg/L) for 5 seconds and stained with eosin G (5 seconds) and thiazine (5 seconds) (Diff-Quik, Baxter Dade Ltd, Switzerland) in order to differentiate eosinophils, neutrophils, macrophages and lymphocytes. A total of 500 cells per smear were counted by light microscopy under oil immersion (x 1000).

Results

Ovalbumin challenge resulted in a significant increase in eosinophils, neutrophils, and total leukocytes in BAS fluid from actively sensitized, vehicle treated (V) Brown Norway rats as compared to naive, untreated (N) rats. At doses of 0.03 mg/kg (A) and 0.1 mg/kg (B), 3b failed to prevent this increase. At a dose of 0.1 mg/kg (C), budesonide also had not effect when administered alone. However, the combination of 0.1 mg/kg budesonide with 3b at 0.03 mg/kg (D) or 0.1 mg/kg (E) produced a significant reduction in eosinophil count. Statistically significant reduction in neutrophil count was achieved only in the 0.1/0.03 mg/kg (D) group. At higher dose (0.5 mg/kg, group F) budesonide treatment alone was effective in preventing the increase in eosinophils, neutrophils, and total leukocytes, and combination of budesonide (0.5 mg/kg) with 0.03 mg/kg (G) or 0.1 mg/kg (H) of 3b was also efficacious (FIGS. 5-6).

Conclusion

The combination of compound 3b with the glucocorticoid budesonide is effective in preventing leukocyte influx following allergen challenge in an animal model of asthma at doses where neither drug alone has any effect.

### Example 4

### Preparation offormyl amides 14 (Scheme 1)

Acyl oxazolidinone 9b (R=n-Pr)

A cooled (−78 ° C.) solution of (S)-(-)-4-benzyl-2-oxazolidinone (4.0 g, 22.6 mmol) in 75 mL anhydrous THF was treated with a 2.5 M solution of n-BuLi in hexane (9.1 mL, 22.6 mmol) over 15 min. After 5 min, neat valeryl chloride (2.95 nL, 24.9 mmol) was added dropwise and the mixture was stirred for another 45 min. at −78 ° C. The mixture was then allowed to reach room temperature, stirred for another 90 min, and then treated with 50 mL saturated NH₄Cl solution. Dichloromethane (50 mL) was then added and the organic layer was washed with brine (2×30 mL), dried over MgSO₄ and concentrated in vacuo. This afforded 5.94 g (100%) of the desired acyl oxazolidinone 9b as a clear colorless oil. ¹H NMR (300 MHz, CDCl₃) δ6 7.36-7.20 (m, 5 H), 4.71-4.64 (rn, 1 H), 4.23-4.14 (m, iH), 3.40 (dd, J=13.3, 3.2 Hz, 1 H), 3.04-2.84 (m, 2 H), 2.77 (dd, J=13.3, 9.6 Hz, 1 H), 1.74-1.63 (m, 2 H), 1.46-1.38 (m, 2 H), 0.96 (t, J=7.3 Hz, 3 H).

Acyl oxazolidinone 9a (R=Et)

By a procedure analogous to that described for preparing acyl oxazolidinone 9b, the lithium anion of (S)-(-)-4-benzyl-2-oxazolidinone was treated with butyryl chloride to provide acyl oxazolidinone 9a in 94% yield. ¹H NMR (300 MHz, CDCl₃) δ7.37-7.20 (m, 5 H), 4.68 (ddd, J=13.1, 7.0, 3.4 Hz, 1 H), 4.23-4.13 (m, 2 H), 3.30 (dd, J=13.3, 9.6 Hz, 1 H), 3.02-2.82 (m, 2 H), 2.77 (dd, J=13.3, 9.6 Hz, 1 H), 1.73 (q, J=7.3 Hz, 2 H), 1.01 (t, J=7.3 Hz, 3 H).

Acyl oxazolidinone 9c (R=n-Bu)

By a procedure analogous to that described for preparing acyl oxazolidinone 9b, the lithium anion of (S)-(-)-4-benzyl-2-oxazolidinone was treated with hexanoyl chloride to provide acyl oxazolidinone 9a in 96% yield. ¹H NMR (300 MHz, CDCl₃) δ7.36-7.20 (m, 5 H), 4.68 (m, 1 H), 4.23-4.14 (m, 2 H), 3.30 (dd, J=13.3, 3.3 Hz, 1 H), 3.02-2.83 (m, 2 H), 2.76 (dd, J=13.3, 9.6 Hz, 1 H), 1.70 (m, 2 H), 1.43-1.34 (m, 4 H), 0.92 (t, J=3.3 Hz, 3 H).

4-Methylvaleryl chloride

4- Methylvaleryl chloride was prepared from commercially available 4-methylvaleric acid in the following way: a cold (0° C.) solution of 4-methylvaleric acid (1.85 mL, 15.0 mmol) in 50 mL anhydrous CH₂Cl₂ containing 10 mL of DMF was treated with 1.95 μL oxalyl chloride (22.5 nmmol). The mixture was then stirred for 3 h at room temperature, concentrated in vacuo and filtered to affords 1.65 g (100%) of the desired acid chloride as a colorless liquid.

Acyl oxazolidinone 9d (R=i-Bu)

By a procedure analogous to that described for preparing acyl oxazolidinone 9b, the lithium anion of (S)-(-)-4-benzyl-2-oxazolidinone was treated with 4-methylvaleryl chloride to provide acyl oxazolidinone 9d in 85% yield. ¹H NMR (300 MHz, CDCl₃) δ7.37-7.20 (m, 5 H), 4.70-4.63 (m, 1 H), 4.23-4.15 (m, 2 H), 3.30 (dd, J=13.2, 3.2 Hz, 1 H), 2.98-2.90 (m, 2 H), 2.76 (dd, J=13.3, 9.6 Hz, 1 H), 1.68-1.54 (m, 3 H), 0.94 (d, J=6.2 Hz, 3 H).

Acyl oxazolidinone 9e (R=CH₂Ph)

By a procedure analogous to that described for preparing acyl oxazolidinone 9b, the lithium anion of (S)-(-)-4-benzyl-2-oxazolidinone was treated with hydrocinnamoyl chloride to provide acyl oxazolidinone 9e in 82% yield. ¹H NMR (300 MHz, CDCl₃) δ7.35-7.16 (m, 10 H), 4.70-4.63 (m, 1 H), 4.21-4.14 (m, 2 H), 3.38-3.19 (m, 3 H), 3.08-2.98 (m, 2 H), 2.75 (dd, J=13.4, 9.5 Hz, 1 H).

Acyl oxazolidinone 10b (R=n-Pr)

A cold (0° C.) solution of acyl oxazolidinone 9b (5.74 g, 22.0 mmol) in 110 mL anhydrous CH₂Cl₂ was treated with 2.52 mL TiCl₄ (23.1 mmol) resulting in the formation of an abundant precipitate. After 5 min, diisopropylethylamine (4.22 mL, 24.2 mmol) was added slowly and the resulting dark brown solution was stirred at room temperature for 35 min. Benzyl chloromethyl ether (6.0 mL, 44.0 mmol) was the rapidly added and the mixture was stirred for 5 h at room temperature. 50 mL CH₂Cl₂ and 75 mL of 10% aqueous NH₄Cl were then resulting in the formation of yellow gummy material. After stirring the suspension vigorously for 10 min, the supernatant was transferred in a separatory funnel and the gummy residue was taken up in 100 mL 1:1 10% aqueous NH₄CI/CH₂Cl₂. The combined organic layers were then washed successively with 1N aqueous HCl, saturated NaHCO₃ and brine, dried over MgSO₄ and concentrated in vacuo. The crude solid material was recrystallized from EtOAc/hexane affording 6.80 g of desired acyl oxazolidinone 10b as a white solid in 81% yield. ¹H NMR (300 MHz, CDCl₃) δ7.34-7.18 (m, 10 H), 4.77-4.69 (m, 1 H), 4.55 (s, 2 H), 4.32-4.23 (m, 1 H), 4.21-4.10 (m, 2 H), 3.80 (t, J=9.0 Hz, 1 H), 3.65 (dd, J=9.0, 5.0 Hz, 1 H), 3.23 (dd, J=13.5, 3.3 Hz, 1 H), 2.69 (dd, J=13.5, 9.3 Hz, 1 H), 1.74-1.64 (m, 1 H), 1.54-1.44 (m, 1 H), 1.40-1.28 (m, 2 H), 0.91 (t, J=7.3 Hz, 3 H).

LRMS (FAB) m/e 382 (M+H⁺)

Acyl oxazolidinone 10a (R=Et)

By a procedure analogous to that described for preparing acyl oxazolidinone 10b, acyl oxazolidinone 10a was obtained in 80% yield. ¹H NMR (300 MHz, CDCl₃) δ7.36-7.18 (m, 10 H), 4.55 (s, 2 H), 4.21-4.11 (m, 3 H), 3.81 (t, J=9.0 Hz, 1 H), 3.66 (dd, J=9.0, 5.0 Hz, 1 H), 3.23 (dd, J=13.5, 3.2 Hz, 1 H), 2.70 (dd, J=13.5, 9.3 Hz, 1 H), 1.78-1.57 (m, 2 H), 0.94 (t, J=7.5 Hz, 3 H).

Acyl oxazolidinone 10c (R=n-Bu)

By a procedure analogous to that described for preparing acyl oxazolidinone 10b, acyl oxazolidinone 10c was obtained in 91% yield. ¹H NMR (300 MHz, CDCl₃) δ7.38-7.17 (m, 10 H), 4.72 (m, 1 H), 4.54 (s, 2 H), 4.27-4.10 (m, 2 H), 3.79 (t, J=8.7 Hz, 1 H), 3.65 (dd, J=9.1, 5.0 Hz, 1 H), 3.23 (dd, J=13.5, 3.3 Hz, 1 H), 2.68 (dd, J=13.5, 9.3 Hz, 1 H), 1.75-1.68 (m, 1 H), 1.31-1.26 (m, 4 H), 0.87 (t, J=6.8 Hz, 3 H).

Acyl oxazolidinone 10d (R=i-Bu)

By a procedure analogous to that described for preparing acyl oxazolidinone 10b, acyl oxazolidinone 10d was obtained in 98% yield. ¹H NMR (300 MHz, CDCl₃) δ7.38-7.17 (m, 10 H), 4.75-4.67 (m, 1 H), 4.57 (d, J=12.0 Hz, 1 H), 4.51 (d, J=12.0 Hz, 1 H), 4.41-4.36 (m, 1 H), 4.20-4.09 (m, 2 H), 3.74 (t, J=9.0 Hz, 1 H), 3.65 (dd, J=9.0, 5.1 Hz, 1 H), 3.23 (dd, J=13.5, 3.2 Hz, 1 H), 2.63 (dd, J=13.5, 9.5 Hz, 1 H), 1.74-1.52 (m, 2 H), 1.35 (dd, J=13.1, 6.1 Hz, 1 H), 0.92 (d, J=2.9 Hz, 3 H), 0.90 (d, J=2.9 Hz, 3 H).

Acyl oxazolidinone 10e (R=CH₂Ph)

By a procedure analogous to that described for preparing acyl oxazolidinone 10b, acyl oxazoidinone 10e was obtained in 84% yield. ¹H NMR (300 MHz, CDCl₃) δ7.38-7.15 (m, 15 H), 4.62-4.50 (m, 4 H), 4.03 (dd, J=9.0, 2.7 Hz, 1 H), 3.93-3.82 (m, 2 H), 3.66 (dd, J=9.2, 4.8 Hz, 1 H), 3.19 (dd, J=13.5, 3.2 Hz, 1 H), 2.98 (dd, J=13.4, 8.2 Hz, 1 H), 2.88 (dd, J=13.4, 7.3 Hz, 1 H), 2.68 (dd, J=13.5, 9.3 Hz, 1 H).

Carboxylic acid 11b (R=n-Pr)

A cold (0° C.) solution of 6.60 g (17.3 mmol) of acyl oxazolidinone 10b in 320 mL THF/H₂O was treated successively with 6.95 mL 35% aqueous H₂O₂ and a solution of lithium hydroxide monohydrate (1.46 g, 34.6 mmol) in 20 mL H₂O. The mixture was stirred for 16 h at 0° C. and then treated carefully first with a solution Na₂SO₃ (10.5 g) in 55 mL H₂O and then with a solution of NaHCO₃ (4.35 g) in 100 mL H₂O. The mixture was stirred for 30 min at room temperature and concentrated in vacuo to remove the THF. The resulting aqueous mixture was then washed with CH₂Cl₂ (4×75 mL), cooled to 0° C., acidified with 6N aqueous HCl and extracted with CH₂Cl₂ (1×200 mL and 3×100 mL). The combined organic layers were then dried over MgSO₄ and concentrated in vacuo affording 3.47 g (90%) of desired acid 11b as a clear colorless oil. ¹H NMR (300 MHz, CDCl₃) δ7.38-7.26 (m, 5 H), 4.55 (s, 2 H), 3.67 (m, 1 H), 3.57 (dd, J=9.2, 5.2 Hz, 1 H), 2.75 (m, 1 H), 1.72-1.31 (m, 4 H), 0.93 (t, J=7.2 Hz, 3 H).

LRMS (FAB) m/e 223 (M+H⁺)

Carboxylic acid 11a (R=Et)

By a procedure analogous to that described for preparing acyl oxazolidinone 11b, acyl oxazolidinone 11a was obtained in 48% yield. ¹H NMR (300 MHz, CDCl₃) δ7.36-7.27 (m, 5 H), 4.55 (s, 2 H), 3.68 (dd, J=9.2, 7.9 Hz, 1 H), 3.59 (dd, J=9.2, 5.4 Hz, 11 H), 2.68-2.65 (m, 1 H), 1.71-1.62 (m, 2 H), 0.97 (t, J=7.5 Hz, 3 H).

Carboxylic acid 11c (R=n-Bu)

By a procedure analogous to that described for preparing acyl oxazolidinone 11b, acyl oxazolidinone 11c was obtained in 96% yield. ¹H NMR (300 MHz, CDCl₃) δ7.37-7.28 (m, 5 H), 4.55 (s, 2 H), 3.67 (dd, J=9.1, 8.1 Hz, 1 H), 3.57 (dd, J=9.2, 5.3 Hz, 1 H), 2.72 (m, 11 H), 1.67-1.51 (m, 2 H), 1.36-1.27 (m, 4 H), 0.89 (t, J=6.9 Hz, 3 H).

Carboxylic acid 11d (R=i-Bu)

By a procedure analogous to that described for preparing acyl oxazolidinone 11b, acyl oxazolidinone 11d was obtained in 80% yield. ¹H NMR (300 MHz, CDCl₃) δ7.37-7.28 (m, 5 H), 4.55 (s, 2 H), 3.64 (t, J=9.1 Hz, 1 H), 3.54 (dd, J=9.1, 5.1 Hz, 1 H), 2.81 (m, 1 H), 1.68-1.54 (m, 2 H), 1.36-1.27 (m, 1 H), 0.92 (d, J=4.9 Hz, 3 H), 0.90 (d, J=4.9 Hz, 3 H).

Carboxylic acid 11e (R=CH₂Ph)

By a procedure analogous to that described for preparing acyl oxazolidinone 11b, acyl oxazolidinone 11e was obtained in 92% yield. ¹H NMR (300 MHz, CDCl₃) δ7.38-7.16 (m, 10 H), 4.53 (d, J=12.1 Hz, 1 H), 4.50 (d, J=12.1 Hz, 1 H), 3.68-3.57 (m, 2 H), 3.09-2.85 (m, 3 H).

Diethylamide 12b (R=n-Pr)

A cooled solution (0° C.) of carboxylic acid 11b (3.40 g, 15.3 mmol) in 1:1 MeCN/CH₂Cl₂ (150 mL), containing diethylamine (2.36 mL, 23.0 mmol) and 2-(1 H-benzotriazol-1-yl)- 1,1,3,3-tetramethyluronium tetrafluoroborate (TBTU, 5.89 g, 18.4 mmol), was treated with diisopropylethylamine (6.7 mL, 38.2 mmol) over 1.5 h (syringe pump). The mixture was then concentrated in vacuo and partitioned between ether (200 mL) and H2O (100 mL). The aqueous layer was extracted with more ether (2×100 mL) and the combined organic layers were washed with aqueous 1N HCl (3×50 mL), saturated aqueous NaHCO₃ and brine, dried over MgSO₄ and concentrated in vacuo. Chromatographic purification (230-400 mesh SiO₂, elution with 1:3 AcOEt/hexane) afforded 4.24 g (97%) of diethyl amide 12b as a clear colorless oil. ¹H NMR (300 MHz, CDCl₃) δ7.35-7.23 (m, 5 H), 4.52 (d, J=12.0 Hz, 1 H), 4.44 (d, J=12.0 Hz, 1 H), 3.67 (t, J=8.6 Hz, 1 H), 3.51 (dd, J=8.7, 5.5 Hz, 1 H), 3.46-3.27 (m, 4 H), 2.96 (m, 1 H), 1.67-1.57 (m, 1 H), 1.48-1.22 (m, 4 H), 1.20-1.10 (m, 6H), 0.90 (t, J=7.2Hz,3 H).

LRMS (FAB) m/e 278 (M+H⁺)

Diethylamide 12a (R=Et)

By a procedure analogous to that described for preparing diethylamide 12b, diethylamide 12a was obtained in 73% yield. ¹H NMR (300 MHz, CDCl₃) δ7.33-7.26 (m, 5 H), 4.52 (d, J=12.0 Hz, 1 H), 4.44 (d, J=12.0 Hz, 1 H), 3.68 (t, J=8.6 Hz, 1 H), 3.53-3.33 (m, 5 H), 2.90 (m, 1 H), 1.75-1.50 (m, 2 H), 1.18 (t, J=7.1 Hz, 3 H), 1.13 (t, J=7.1 Hz, 3 H), 0.89 (t, J=7.4 Hz, 3 H).

Diethylamnide 12c (R=n-Bu)

By a procedure analogous to that described for preparing diethylamnide 12b, diethylamide 12c was obtained in 94% yield. ¹H NMR (300 MHz, CDCl₃) δ7.35-7.25 (m, 5 H), 4.51 (d, J=12.0 Hz, 1 H), 4.44 (d, J=12.0 Hz, 1 H), 3.67 (t, J=8.6 Hz, 1 H), 3.51 (dd, J=8.8, 5.5 Hz, 1 H), 3.46-3.29 (m, 1 H), 2.94 (m, 1 H), 1.66-1.62 (m, 2 H), 1.33-1.10 (m, 9 H), 0.85 (t, J=7.0 Hz, 3 H).

Diethylamide 12d (R=i-Bu)

By a procedure analogous to that described for preparing diethylamide 22b, diethylamide 12d was obtained in 95% yield. ¹H NMR (300 MHz, CDCl₃) δ7.35-7.23 (m, 5 H), 4.51 (d, J=12.0 Hz, 1 H), 4.44 (d, J=12.0 Hz, 1 H), 3.65 (t, J=8.7 Hz, 1 H), 3.54-3.28 (mD, 5 H), 3.03 (m, 1 H), 1.63-1.49 (m, 2 H), 1.33-1.24 (mn, 1 H), 1.18 (t, J=7.1 Hz, 3 H), 1.12 (t, J=7.1 Hz, 3 H), 0.90 (t, J=6.4 Hz, 3 H).

Diethylamide 12e (R=CH₂Ph)

By a procedure analogous to that described for preparing diethylamide 12b, diethylamide 12e was obtained in 89% yield. ¹H NMR (300 MHz, CDCl₃) δ7.35-7.16 (m, 10 H), 4.53 (d, J=12.1 Hz, 1 H), 4.47 (d, J=12.1 Hz, 1 H), 3.77 (t, J=8.5 Hz, 1 H), 3.59 (dd, J=8.8, 5.7 Hz, 1 H), 3.40 (m, 1 H), 3.22-2.89 (m, 5 H), 2.79 (dd, J=13.0, 5.1 Hz, 3 H), 1.01 (t, J=7.1 Hz, 3 H), 0.85 (t, J=7.2 Hz, 3 H).

Alcohol 13b (R=n-Pr)

To a solution of diethylamide 12b (4.08 g, 14.7 mmol) in 140 mL MeOH was added 20% Pd(OH)₂/C (400 mg) and the suspension was hydrogenated at atmospheric pressure and room temperature for 15 h. Filtration of the catalyst and concentrating the filtrate in vacuo afforded 2.84 g (100%) of the desired primary alcohol 13b. ¹H NMR (300 MHz, CDCl₃) δ3.74 (br. d, J=4.2 Hz, 1 H), 3.61-3.15 (m, 5 H), 2.71 (m, 1 H), 1.69-1.24 (m, 4 H), 1.20 (t, J=7.1 Hz, 3 H), 1.12 (t, J=7.1 Hz, 3 H), 0.92 (t, J=7.2 Hz, 3 H).

LRMS (FAB) m/e 188 (M+H⁺)

Alcohol 13a (R=Et)

By a procedure analogous to that described for preparing alcohol 13b, alcohol 13a was obtained in 100% yield. ¹H NMR (300 MHz, CDCl₃) δ3.76 (m, 2 H), 3.58-3.19 (m, 4 H), 2.64 (m, 1 H), 1.71-1.65 (m, 2 H), 1.21 (t, J=7.1 Hz, 3 H), 1.13 (t, J=7.1 Hz, 3 H), 0.96 (t, J=7.4 Hz, 3 H).

Alcohol 13c (R=n-Bu)

By a procedure analogous to that described for preparing alcohol 13b, alcohol 13c was obtained in 100% yield. ¹H NMR (300 MHz, CDCl₃) δ3.76 (d, J=4.5 Hz, 2 H), 3.58-3.19 (m, 4 H), 2.72-2.65 (m, 2 H), 1.68-1.55 (m, 2 H), 1.40-1.24 (m, 4 H), 1.20 (t, J=7.1 Hz, 3 H), 1.12 (t, J=7.1 Hz, 3 H), 0.90 (t, J=6.9 Hz, 3 H).

Alcohol 13d (R=i-Bu)

By a procedure analogous to that described for preparing alcohol 13b, alcohol 13d was obtained in 100% yield. ¹H NMR (300 MHz, CDCl₃) δ3.78-3.68 (m, 2 H), 3.57-3.15 (m, 4 H), 2.81-2.73 (m, 1 H), 1.70-1.60 (m, 2 H), 1.40-1.28 (m, 1 H), 1.21 (t, J=7.1 Hz, 3 H), 1.12 (t, J=7.1 Hz, 3 H), 0.92 (m, 6 H).

Alcohol 13e (R=CH₂Ph)

By a procedure analogous to that described for preparing alcohol 13b, alcohol 13e was obtained in 100% yield. ¹H NMR (300 MHz, CDCl₃) δ7.29-7.16 (m, 5 H), 3.81-3.71 (m, 2 H), 3.61-3.50 (m, 1 H), 3.15-2.87 (m, 6 H), 1.05 (t, J=7.1 Hz, 3 H), 0.98 (t, J=7.1 Hz, 3 H).

Aldehyde 14b (R=n-Pr)

To a solution of alcohol 13b (2.34 g, 12.7 mmol) in wet CH₂Cl₂ (125 mL, prepared by stirring CH₂Cl₂ with water and separating the organic layer) was added Dess-Martin periodinane (8.06 g, 19.0 mmol). The mixture was stirred at room temperature for 40 min and was then poured into a mixture of 5% aqueous Na₂S₂O₃ (250 mL) containing 5.2 g NaHCO₃, and ether (200 mL). The biphasic mixture was stirred vigorously for 5 min and the aqueous layer was extracted with 15% CH₂Cl₂/Et₂O (2×100 mL). The combined organic layers were then washed with H₂O (3×75 mL) and brine, dried over MgSO₄, filtered and concentrated in vacuo to afford 2.06 g (88%) of desired aldehyde 14b, a clear colorless oil. ¹H NMR (300 MHz, CDCl₃) δ9.60 (d, J=3.5 Hz, 1 H), 3.49-3.30 (m, 5 H), 1.96-1.85 (m, 2 H), 1.39-1.31 (m, 2 H), 1.19 (t, J=7.1 Hz, 3 H), 1.13 (t, J=7.1 Hz, 3 H), 0.95 (t, J=7.3 Hz, 3 H).

Aldehyde 14a (R=Et)

By a procedure analogous to that described for preparing alcohol 14b, aldehyde 14a was obtained in 80% yield. ¹H NMR (300 MHz, CDCl₃) δ9.61 (d, J=3.6 Hz, 1 H), 3.48-3.29 (m, 5 H), 2.02-1.90 (m, 2 H), 1.19 (t, J=7.1 Hz, 3 H), 1.14 (t, J=7.1 Hz, 3 H), 0.96 (t, J=7.4 Hz, 3 H).

Aldehyde 14c (R=n-Bu)

By a procedure analogous to that described for preparing alcohol 14b, aldehyde 14c was obtained in 98% yield. ¹H NMR (300 MHz, CDCl₃) δ9.59 (d, J=3.6 Hz, 1 H), 3.48-3.29 (m, 5 H), 1.97-1.87 (m, 2 H), 1.39-1.22 (m, 4 H), 1.18 (t, J=7.2 Hz, 3 H), 1.13 (t, J=7.2 Hz, 3 H), 0.90 (t, J=7.0 Hz, 3 H).

Aldehyde 14d (R=i-Bu)

By a procedure analogous to that described for preparing alcohol 14b, aldehyde 14d was obtained in 96% yield. ¹H NMR (300 MHz, CDCl₃) δ9.57 (d, J=3.7 Hz, 1 H), 3.51-3.27 (m, 5 H), 1.83 (t, J=7.1 Hz, 3 H), 1.66-1.55 (m, 1 H), 1.20 (t, J=7.1 Hz, 3 H), 1.13 (t, J=7.1 Hz, 3 H), 0.93 (d, J=6.6 Hz, 6 H).

Aldehyde 14e (R=CH₂Ph)

By a procedure analogous to that described for preparing alcohol 14b, aldehyde 14e was obtained in 97% yield. ¹H NMR (300 MHz, CDCl₃) δ9.69 (d, J=2.9 Hz, 1 H), 7.29-7.16 (m, 5 H), 3.65 (m, 1 H), 3.53-3.42 (m, 1 H), 3.30 (dd, J=13.5, 9.3 Hz, 1 H), 3.23-3.13 (m, 2 H), 3.06-2.91 (m, 2 H), 1.04 (t, J=7.1 Hz, 3 H), 0.93 (t, J=7.1 Hz, 3 H).

### Example 5

### Preparation of β-lactones 3 (Scheme 2)

Aldol 5b (R=n-Pr)

To a cold (−78° C.) solution of trans-oxazoline 4 in ether (35 mL) was added lithium bis(trimethylsilyl)amide (2.17 of a 1 M solution in hexane, 2.17 mmol). After 30 min, the orange solution was treated dropwise with a 1M solution of dimethylaluminum chloride in hexane (4.55 mL, 4.55 mmol) and the mixture was stirred for another 60 min before being cooled down to −85° C. (liquid N₂ was added to the dry ice/acetone bath). A solution of aldehyde 14b (420 mg, 2.27 mmol) in ether (4 mL) was the added over 10 min along the side of the flask. The mixture was then allowed to warm up to −40° C. over 2.5 h and then quenched by adding 35 mL of saturated aqueous NH₄Cl and 25 mL AcOEt. Enough 2 N HCl was then added until 2 clear phases are obtained (ca. 15 mL added). The aqueous layer was extracted with AcOEt (2×20 mL) and the combined organic layers were washed successively with 0.5 N aqueous HCl (20 mL), H₂O (20 mL), 0.5 M aqueous NaHSO₃ (2×15 mL), saturated aqueous NaHCO₃ and finally with brine, then dried over Na₂SO₄ and concentrated in vacuo affording 879 mg (>100%) of crude aldol product 5b which was pure enough to be used directly in the subsequent step. ¹H NMR (300 MHz, CDCl₃) δ8.02-7.97 and 7.53-7.39 (m, 5 H), 6.58 (d, J=9.9 Hz, 1 H), 4.82 (d, J=2.4 Hz, 1 H), 3.73 (s, 3 H), 3.69-3.61 (m, 2 H), 3.49-3.39 (m, 2 H), 3.24-3.16 (m, 1 H), 3.05 (m, 1 H), 2.89 (m, 1 H), 2.28-2.23 (m, 1 H), 1.98-1.91 (m, 1 H), 1.37-1.20 (m, 6 H), 1.19-1.06 (m, 6 H), 0.87 (t, J=7.1 Hz, 3 H), 0.70 (d, J=6.7 Hz, 3 H).

Aldol product 5b was also obtained in 100% yield by a procedure analogous to that described above but using cis-oxazoline 21 (see below) instead of trans-oxazoline 4.

Aldol 5a (R=Et)

By a procedure analogous to that described for preparing aldol 5b, the lithium anion of trans-oxazoline 4 was treated successively with dimethylaluminum chloride and aldehyde 14a to provide aldol 5a in 95% yield. ¹H NMR (300 MHz, CDCl₃) δ8.00-7.97 and 7.51-7.39 (m, 5 H), 6.50 (d, J=9.9 Hz, 1 H), 4.80 (d, J=2.4 Hz, 1 H), 3.81-3.64 (m, 2 H), 3.74 (s, 3 H), 3.45 (m, 2 H), 3.19 (mn, 2 H), 2.93-2.84 (m, 2 H), 2.24 (m, 1 H), 1.89 (m, 1 H), 1.73-1.64 (m, 4 H), 1.29 (t, J=7.2 Hz, 3 H), 1.12 (d, J=6.9 Hz, 3 H), 1.07 (d, J=7.2 Hz, 3 H), 0.70 (d, J=6.7 Hz, 3 H).

Aldol 5c (R=n-Bu)

By a procedure analogous to that described for preparing aldol 5b, the lithium anion of trans-oxazoline 4 was treated successively with dimethylalurninum chloride and aldehyde 14c to provide aldol 5c in 100% yield. ¹H NMR (300 MHz, CDCl₃) δ8.02-7.98 and 7.53-7.33 (m, 5 H), 6.57 (d, J=10.0 Hz, 1 H), 4.81 (d, J=2.3 Hz, 1 H), 3.73 (s, 3 H), 3.68-3.60 (m, 2 H), 3.49-3.17 (m, 2 H), 3.00 (m, 1 H), 2.90 (m, 1 H), 1.98-1.87 (m, 2 H), 1.38-0.83 (m, 16 H), 0.70 (d, J=6.7 Hz, 3 H).

Aldol 5d (R=i-Bu)

By a procedure analogous to that described for preparing aldol 5b, the lithium anion of trans-oxazoline 4 was treated successively with dimethylaluminum chloride and aldehyde 14d to provide aldol 5d in 100% yield. ¹H NMR (300 MHz, CDCl₃) δ8.01-7.80 and 7.55-7.20 (m, 5 H), 4.87 (d, J=2.3 Hz, 1 H), 3.73 (s, 3 H), 3.69-3.58 (m, 2 H), 3.51-3.32 (m, 2 H), 2.98-2.87 (m, 1 H), 2.33-2.24 (m, 1 H), 2.12-2.02 (m, 1 H), 1.83 (t, J=7.1 Hz, 1 H), 1.35 (t, J=7.1 Hz, 3 H), 1.25-1.05 (m, 5 H), 0.93 (d, J=6.6 Hz, 3 H), 0.89 (d, J=6.5 Hz, 3 H), 0.80 (d, J=6.5 Hz, 3 H), 0.69 (d, J=6.7 Hz, 3 H).

Aldol 5e (R=CH₂Ph)

By a procedure analogous to that described for preparing aldol 5b, the lithium anion of trans-oxazoline 4 was treated successively with dimethylaluminum chloride and aldehyde 14e to provide aldol 5e in 100% yield. ¹H NMR (300 MHz, CDCl₃) δ8.01-7.93 and 7.54-7.10 (m, 10 H), 4.71 (d, J=2.5 Hz, 1 H), 3.73 (s, 3 H), 3.68-3.58 (m, 2 H), 3.48-2.79 (m, 6 H), 2.17 (m, 1 H), 1.12-0.91 (m, 9 H), 0.68 (d, J=6.7 Hz, 3 H).

γ-Lactam 7b (R=n-Pr)

A solution of aldol 5b (4.72 g, 10.9 mmol) in 100 mL 1:9 AcOH/MeOH, to which was added 4.8 g 20% Pd(OH)₂/C, was vigorously shaken under 55 p.s.i. H₂ for 60 h. The mixture was brought down to atmospheric temperature before being filtered and concentrated in vacuo. The solid obtained was purified by flash chromatography (SiO₂, elution with 1% AcOH in 1:1 AcOEt/hexane) affording 2.23 g (75%) of desired γ-lactam 7b as a white solid. ¹H NMR (300 MHz, CDCl₃) δ7.89 (br. s, 1 H), 4.77 (br. d, J=11.5 Hz, 1 H), 4.47 (dd, J=11.5, 5.6 Hz, 1 H), 4.08 (dd, J=9.4, 5.0 Hz, 1 H), 3.83 (s, 3 H), 2.93 (m, 1 H), 1.78-1.39 (rM 6 H), 1.02-0.88 (m, 9H).

γ-Lactam 7a (R=Et)

By a procedure analogous to that described for preparing γ-lactam 7b, aldol 5a was hydrogenated at 55 p.s.i. for 48 h to provide γ-lactam 7a in 72% yield. ¹H NMR (300 MHz, CDCl₃) δ7.79 (br. s, 1 H), 4.62 (br. d, J=11.2 Hz, 1 H), 4.51 (dd, J=11.2, 5.4 Hz, 1 H), 3.83 (s, 3 H), 2.85 (m, 1 H), 1.77-1.64 (m, 3 H), 1.01 (t, J=7.4 Hz, 3 H), 0.98 (d, J=6.9 Hz, 3 H), 0.95 (d, J=6.9 Hz, 3 H).

γ-Lactam 7c (R=n-Bu)

A solution of aldol 5c (361 mg, 0.80 mmol) in 6 mL 1:9 AcOH/MeOH, to which was added 250 mg 20% Pd(OH)₂/C, was vigorously shaken under 50 p.s.i. H₂ for 24 h. More catalyst (100 mg) was then added and the mixture was again shaken at 50 p.s.i. for another 24 h after which time it brought down to atmospheric temperature before being filtered. The filtrate was then heated to reflux for 30 min, cooled to room temperature and concentrated in vacuo. The solid obtained was co-evaporated once with toluene and purified by flash chromatography (SiO₂, elution with 4% MeOH/CHCl₃) affording 140 mg (61%) of desired γ-lactam 7c as a white solid. ¹H NMR (300 MHz, CDCl₃) δ8.02 (br. s, 1 H), 4.93 (br. d, J=11.3 Hz, 1 H), 4.46 (dd, J=11.3, 5.5 Hz, 1 H), 4.15-4.08 (m, 1 H), 3.83 (s, 3 H), 2.94-2.87 (m, 1 H), 1.80-1.34 (m, 6 H), 0.94 (d, J=6.9 Hz, 3 H), 0.89 (t, J=7.2 Hz, 3 H).

γ-Lactam 7d (R=i-Bu)

By a procedure analogous to that described for preparing γ-lactam 7c, aldol 5d was hydrogenated at 50 p.s.i. for 40 h and heated to reflux for 30 min providing γ-lactam 7d in 61% yield. ¹H NMR (300 MHz, CDCl₃) δ7.92 (br. s, 1 H), 4.81 (br. d, J=11.5 Hz, 11 H), 4.46 (m, 1 H), 4.09 (m, 1 H), 3.83 (s, 3 H), 3.04-2.98 (m, 1 H), 1.78-1.73 (m, 2 H), 1.66-1.47 (m, 3 H), 1.00-0.90 (m, 12 H).

γ-Lactam 7e (R=CH₂Ph)

By a procedure analogous to that described for preparing γ-lactam 7c, aldol 5e was hydrogenated at 50 p.s.i. for 24 h and heated to reflux for 30 min providing γ-lactam 7e in 71% yield. ¹H NMR (300 MHz, CDCl₃) δ8.01 (br. s, 1 H), 7.35-7.15 (m, 5 H), 5.02 (br. d, J=11.7 Hz, 1 H), 4.40-4.34 (m, 1 H), 4.06-4.01 (m, 1 H), 3.84 (s, 3 H), 3.34-3.27 (m, 1 H), 3.10-3.04 (m, 2 H), 1.84-1.72 (m, 1 H), 0.98 (d, J=6.7 Hz, 3 H), 0.93 (d, J=6.9 Hz, 3 H).

β-Lactone 3b (R=n−Pr; 7-n-propyl-clasto-lactacystin β-lactone)

To a cold (0° C.) solution of γ-lactam 7b (2.20 g, 8.06 mmol) in EtOH (100 mL) was added 0.1N aqueous NaOH (100 mL, 10.0 mmol). The mixture was stirred at room temperature for 15 h after which time H2O (50 mL) and AcOEt (100 mL) were added. The aqueous layer was then washed with AcOEt (2×50 mL), acidified with 6N aqueous HCl and concentrated in vacuo to a volume of ca 60 mL. This solution was then frozen and lyophilized. The obtained solid was suspended in THF, filtered to get rid of sodium chloride and concentrated in vacuo affording 2.05 g (98%) of the desired dihydroxyacid as white solid. ¹H NMR (300 MHz, CD₃OD) δ4.42 (d, J=5.8 Hz, 1 H), 3.90 (d, J=6.5 Hz, 1 H), 2.84 (m, 1 H), 1.70-1.24 (m, 6 H), 0.95-0.84 (m, 9 H).

To a solution of the dihydroxyacid (1.90 g, 7.33 mmol) in anhydrous THF (36 mL) was added a solution of 2-(1H-benzotriazol-1-yl)-1,1,3,3-tetramethyluronium tetrafluoroborate (TBTU, 2.59, 8.06 mmol) in anhydrous MeCN (36 mL) followed by triethylamine (0.72 mL, 22.0 mmol). After stirring for 70 min at room temperature, some toluene was added and the mixture was concentrated in vacuo and co-evaporated 2 more times with toluene. Purification by flash chromatography (SiO₂, elution with 2:3 AcOEt/hexane) afforded 1.44 g (81%) of desired β-lactone 3b as a white solid. ¹H NMR (300 MHz, CDCl₃) δ6.07 (br. s, 1 H), 5.26 (d, J=6.1 Hz, 1 H), 3.97 (dd, J=6.4, 4.4 Hz, 1 H), 2.70-2.63 (m, 1 H), 2.03 (d, J=6.4 Hz, 3 H), 1.93-1.44 (m, 5 H), 1.07 (d, J=7.0 Hz, 3 H), 0.99 (d, J=7.3 Hz, 3 H), 0.91 (d, J=6.7 Hz, 3 H).

LRMS (FAB) m/e 242 (M+H⁺)

β-Lactone 3a (R=Et; 7-ethyl-clasto-lactacystin β-lactone)

Hydrolysis of 7a, as described for 7b above, afforded the corresponding dihydroxyacid in 100% yield. ¹H NMR (300 MHz, CD₃OD) δ4.45 (d, J=5.8 Hz, 1 H), 3.90 (d, J=6.4 Hz, 1 H), 2.74 (m, 1 H), 1.71-1.53 (m, 3 H), 0.94 (t, J=7.4 Hz, 3 H), 0.92 (d, J=6.8 Hz, 3 H), 0.88 (d, J=6.8 Hz, 3 H).

By a procedure analogous to that described for preparing β-lactone 3b, β-lactone 3a was obtained in 79% yield. ¹H NMR (300 MHz, CDCl₃) δ6.17 (br. s, 1 H), 5.30 (d, J=6.0 Hz, 1 H), 3.98 (dd, J=6.4, 4.4 Hz, 1 H), 2.60 (m, 1 H), 2.08 (d, J=6.4 Hz, 3 H), 1.97 (m, 2 H), 1.75 (m, 1 H), 1.12 (t, J=7.5 Hz, 3 H), 1.07 (d, J=6.8 Hz, 3 H), 0.92 (d, J=6.8 Hz, 3 H).

β-Lactone 3c (R=n-Bu; 7-n-butyl-clasto-lactacystin β-lactone)

Hydrolysis of 7c, as described for 7b above, afforded the corresponding dihydroxyacid in 100% yield. ¹H NMR (300 MHz, CD₃OD) δ4.42 (d, J=5.8 Hz, 1 H), 3.90 (d, J=6.4 Hz, 1 H), 2.86-2.79 (m, 1 H), 1.70-1.24 (m, 8H), 0.97-0.86 (m, 9 H).

By a procedure analogous to that described for preparing β-lactone 3b, β-lactone 3c was obtained in 40% yield. ¹H NMR (300 MHz, CDCl₃) δ6.14 (br. s, 1 H), 5.27 (d, J=6.1 Hz, 1 H), 3.97 (d, J=4.4 Hz, 1 H), 2.68-2.61 (m, 1 H), 1.94-1.86 (m, 2 H), 1.72-1.36 (m, 7 H), 1.07 (d, J=7.0 Hz, 3 H), 0.93 (t, J=7.1 Hz, 3 H), 0.91 (d, J=6.8 Hz, 3 H).

LRMS (FAB) m/e 256 (M+H⁺)

β-Lactone 3d (R=i-Bu; 7-i-butyl-clasto-lactacystin β-lactone)

Hydrolysis of 7d, as described for 7b above, afforded the corresponding dihydroxyacid in 100% yield. ¹H NMR (300 MHz, CD₃OD) δ4.50 (d, J=5.8 Hz, 1 H), 4.00 H (d, J=6.5 Hz, 1 H), 3.09-3.02 (m, 1 H), 1.90-1.61 (m, 3 H), 1.49-1.40 (m, 2 H), 1.02 (d, J=6.7 Hz, 3 H), 0.98 (d, J=6.5 Hz, 3 H), 0.97 (d, J=6.7 Hz, 3 H).

By a procedure analogous to that described for preparing β-lactone 3b, β-lactone 3d was obtained in 62% yield. ¹H NMR (300 MHz, CDCl₃) δ6.16 (br. s, 1 H), 5.25 (d, J=6.1 Hz, 1 H), 3.97 (d, J=4.4 Hz, 1 H), 2.71 (dd, J=15.1, 6.2 Hz, 1 H), 1.95-1.66 (m, 5 H), 1.08 (d, J=6.9 Hz, 3 H), 0.99 (d, J=6.3 Hz, 3 H), 0.98 (d, J=6.3 Hz, 3 H), 0.92 (d, J=6.7 Hz, 3 H).

LRMS (FAB) m/e 256 (M+H⁺)

β-Lactone 3e (R=CH₂Ph; 7-benzyl-clasto-lactacystin β-lactone)

Hydrolysis of 7e, as described for 7b above, afforded the corresponding dihydroxyacid in 88% yield. ¹H NMR (300 MHz, CD₃OD) δ7.25-7.04 (m, 5 H), 4.29 (d, J=5.7 Hz, 1 H), 3.83 (d, J=6.4 Hz, 1 H), 3.01-2.82 (m, 3 H), 1.65 (m, 1 H), 0.90 (d, J=6.6 Hz, 3 H), 0.86 (d, J=6.8 Hz, 3 H).

By a procedure analogous to that described for preparing β-lactone 3b, β-lactone 3e was obtained in 77% yield. ¹H NMR (300 MHz, CDCl₃) δ7.36-7.20 (m, 5 H), 6.57 (br. s, 1 H), 5.08 (d, J=5.4 Hz, 1 H), 3.94 (d, J=4.5 Hz, 1 H), 3.25 (d, J=10.1 Hz, 1 H), 3.01-2.89 (m, 2 H), 1.92-1.81 (m, 1 H), 1.05 (d, J=6.9 Hz, 3 H), 0.86 (d, J=6.7 Hz, 3 H).

LRMS (FAB) m/e 290 (M+H⁺)

### Example 6

### Pharmacokinetics of N-(Pyrazine)carbonyl-L-phenylalanine-L-leucine Boronic Acid (1) in Rats and Primates

Rats

A single dose intravenous pharmacokinetics study with N-(pyrazine)carbonyl-L-phenylalanine-L-leucine boronic acid (1) was conducted in Sprague-Dawley rats (140 to 280 g). Animals were assigned to 3 groups (6/sex in Groups 1 and 2; 9/sex in Group 3). Animals in groups 1, 2, and 3 received 0.03, 0.1 or 0.3 mg/kg of 1, respectively, in the same dose volume.

Blood samples (approximately 1.0 mL) were collected from the jugular vein of animals pre-dose and at approximately 10 and 30 min and 1, 3 and 24 h post-dose on Day 1. The samples were assayed for 1 using a chromatography/mass spectroscopy (LC/MS/MS) method. The lower limit of quantitation for analysis was established at 2.5 ng/mL for 1 in rat plasma and whole blood.

Following the single intravenous doses, plasma or whole blood levels of 1 were only measurable at the 0.3 mg/kg dose level. The observed Cₘₐₓ occurred at the first time point; hence, the time to peak concentration (Tₘₐₓ) was estimated to be α 10 min in both male and female rats. Males generally had slightly higher peak concentration (Cₘₐₓ) and area under the concentration-time curve (AUC₀₋ₜ) values than females. The Cₘₐₓ values in plasma and in whole blood in males were 51.8 and 22.7 ng/mL, respectively and in females were 36.9 and 19.1 ng/mL, respectively. The AUC₀₋ₜ values in plasma and whole blood in males were 14.0 and 18.6 ng•h/mL, respectively and in females were 12.9 and 17.7 ng•h/mL, respectively. Estimation of the elimination half-life (t_{½}) was not possible due to the fluctuation of 1 levels during the terminal phase. The observations suggest that 1 is rapidly cleared from the blood.

Primates

Levels of 1 in blood and plasma were measured at 2 hours post-dose in a range-finding study in primates. Single intravenous doses of 1 were administered to two cynomolgus monkeys (1 male, 3.3 kg; 1 female, 2.3 kg). Each monkey received two single doses (0.1 mg/kg on Day 1 and 0.3 mg/kg on Day 8) at a dose volume of 1.0 mL/kg. The vehicle was 0.1% ascorbic acid/2% ethanol/98% saline (0.9%). This work was carried out by Covance Laboratories Inc., Madison, WI.

Following intravenous administration, blood was collected from each animal on Days 1 and 8 at approximately 2 h after dosing. The blood and plasma samples were stored in a freezer set to maintain −20±10° C. until analyzed for test material content.

Samples were assayed for 1 using a chromatography/mass spectroscopy (LC/MS/MS) method. The lower limit of quantitation for analysis was established at 2.5 ng/mL for 1 in monkey plasma and whole blood. Two hours following administration of 0.1 mg/kg of 1, concentrations of 1 were less than 2.5 ng/mL (male and female) in plasma; the concentration of 1 in whole blood was 3.72 ng/mL in the male and 3.86 ng/mL in the female. Two hours following administration of 0.3 mg/kg of 1, concentrations of 1 in plasma were 4.64 ng/mL (female) and 6.44 ng/mL (male); concentrations of 1 in whole blood were 10.6 ng/mL (female) and 9.01 ng/mL (male).

### Example 7

### Preparation of peripheral white blood cell lysates for in vitro measurement of 20 S proteasome activity

This preparation procedure applies to blood samples collected from mammals, particularly rats, mice, dogs, pigs, rabbits, non-human primates, or human subjects. Peripheral white blood cells are separated from blood samples upon collection for storage at about −70° C. until tested. To avoid interference with the assay due to the presence of endogenous proteasome inhibitors, it is important that red blood cells be rigorously excluded.

PROCEDURE

The required amount of blood is collected into a tube containing anticoagulant. For human subjects and primates, approximately 5 mL of blood is required; for rats, approximately 4 mL of blood is needed; for mice, approximately 1 mL of blood is needed from each of five mice, and the five blood samples are pooled to provide approximately 5 mL.

The blood sample is diluted 1:1 (v/v) with sterile saline, and the blood-saline mixture is layered over NYCOPREP^{™} separation medium (GIBCO BRL Products) in a 14×75 mm polystyrene test tube at a ratio of approximately 2:1 blood:NYCOPREP^{™}. The sample is centrifuged at 500 x g for approximately 30 minutes at room temperature. The top layer is removed, leaving ˜2-3 mm of the cell band between the top and bottom layers. The remaining cell band is transferred by pipette to a clean centrifuge tube. The cell band is washed with 3 mL of cold phosphate-buffered saline and centrifuged at 400 x g for 5 minutes at 4° C. The supernatant is poured off and the pellet is resuspended in ˜1 mL of cold phosphate-buffered saline. The suspension is transferred to a 1.5 mL Eppendorf microfuge tube and microfuged at 6600 x g for approximately 10 minutes at 4° C. The supernatant is aspirated off and the cell pellet is stored at −70° C.±10° C.

### Example 8

### Assay to measure 20 S proteasome activity in peripheral white blood cells

### Specific activity method

The assay is based upon the SDS-inducible chymotrypsin-like activity of free 20 S particles. It uses fluorometry to measure the rate at which the 20 S proteasome hydrolyzes an amide bond in a small peptide substrate. Measurement of this rate in the absence and in the presence of an inhibitor allows a determination of how enzyme is bound by inhibitor. This assay is used to measure 20 S proteasome activity in peripheral white blood cells in mammals, particularly rats, mice, dogs, pigs, rabbits, non-human primates, or human subjects.

ABBREVIATIONS AND DEFINITIONS:

AMC 7-amino-4-methylcoumarin

DMF dimethyl formamide

BSA bovine serum albumin

DMSO dimethyl sulfoxide

DTT dithiothreitol

EDTA disodium ethylenediaminetetraacetate

HEPES N-(2-Hydroxyethyl)piperazine-N -(2-ethanesulfonic acid); pH adjustments with NaOH

Hgb hemoglobin

SDS sodium dodecylsulfate of either - SDS-grade: 99% sodium dodecylsulfate Lauryl grade: ˜70% dodecyl sulfate with the remainder as tetradecyl and hexadecyl sulfates.

TMB 3,3′,5,5′-tetramethylbenzidine

WBC white blood cells

Ys substrate N-(N-Succinylleucylleucylvalyltyrosyl)-7-amino-4-methylcoumarin (Suc-Leu-Leu-Val-Tyr-AMC) (Bachem)

MilliQ water water purified by reverse osmosis or ion exchange and further treated with a Millipore MilliQ Plus UF water purifying system (or equivalent system) resulting in water with a resistivity greater than 16 MΩ•cm.

PROCEDURE

The Ys substrate is dissolved to 6 mM in DMSO. A 2% (2 g/100 mL) solution of SDS in MilliQ water is prepared in a glass bottle. The Ys substrate buffer, containing 20 mM HEPES, 0.5 mM EDTA, 0.035% SDS, 1% DMSO, and 60 μM Ys substrate, is prepared. The final pH of the Ys buffer is 8.0.

Purified 20 S proteasome standard from rabbit reticulocytes, prepared according to the literature procedure (McCormack et al., Biochemistry 37:7792-7800 (1998)), is diluted 1:9 (v/v) in 20 mM HEPES/0.5 mM EDTA (pH 7.8).

To 5 μL of a 20 mM AMC stock solution in DMF is added 2 mnL of DMF. The resultant solution is diluted 1:25 in DMSO to produce a 2 μM AMC solution. The zero value for Ys substrate buffer is recorded on a fluorometer (λem=440 nm; λex=380 nm). To 2 mL of Ys substrate buffer is added 5 μL of AMC every 30 seconds for a total of five times to produce a calibration curve for 0 to 50 pmol of AMC. After each addition, a fluorometer reading is taken with an excitation band width of 10 nm and an emission band width of 20 nm. The slope is the fluorometer calibration.

The 20 S proteasome standard is diluted 1:10 in 20 mM HEPES/0.5 mM EDTA (pH 7.8) to form a 12 μg/mL stock solution and placed on ice. 10 μL of the standard 20 S proteasome solution is added to a cuvette containing 2 mL of Ys substrate buffer and the reaction is run for 10 minutes. The maximum linear slope is measured on a fluorometer and provides a calibration of Ys substrate buffer and the assay conditions (Ys calibration). This value is divided by the fluorometer calibration to provide the standardized activity of standard 20 S proteasome.

White blood cells, prepared as described in Example 1, are lysed by adding 200 μL of 5 mM EDTA to each sample. The samples are allowed to stand on ice for at least 15 minutes.

Bradford protein assay (measuring total protein content) and hemoglobin assay are performed on the test sample following standard procedures using commercially available kits. The accurate measure of white blood cell 20 S proteasome activity cannot be determined if the hemoglobin content is greater than 10% that of total protein. In this situation, the sample should be treated as a whole blood cell lysate.

10 μL of a test sample is added to a cuvette containing 2 mL of Ys substrate buffer at 37° C., and the reaction is allowed to run for 10 minutes. Complete activation of the 20 S proteasome is achieved within 10 minutes. Consistent results are obtained for readings taken after 4 minutes and up to 10 minutes. The maximum linear slope for at least 1 minute of data is measured. If the rate is less than 1 pmol AMC/sec, the measurement is repeated using 20 μL of the test sample.

The amount of 20 S proteasome activity in the test sample is calculated according to the following formula:

$$20 S activity = \frac{Rate ( FU / min ) * fluorometer calibration ( pmol / FU )}{\begin{matrix}0.0001 * WBC protein ( mg ) * 60 s / min * \\ Ys calibration ( pmol / s )\end{matrix}}$$

In order for the assay to be considered valid, the hemoglobin present in the sample must be less than 10% of the total protein, and triplicate 20 S proteasome activity values must have a standard deviation of no more than 3%.

### Example 9

### Derivation of equation relating chymotryptic:tryptic activity ratio to percent inhibition by a proteasome inhibitor

Let k_{c} and kₜ be the apparent rate constants for the chymotryptic and tryptic sites, respectively, under standard assay conditions (no inhibitor):

v_{c}=k_{c}[20 S]ₜ (1)

vₜ=kₜ[20 S]ₜ (2)

where [20 S]ₜ=total proteasome concentration.

In the presence of a proteasome modifier that results in formation of an E•I complex, the rate constant for chymotryptic and tryptic sites may be altered by the single molecule of modifier binding to an unidentified site. This effect can be represented by β_{c}k_{c} and βₜkₜ

where β=0 indicates total inhibition by the modifier (i.e., E•I complex has no activity)

β<1 indicates partial inhibition (i.e., E•I complex has less activity than E)

β=1 indicates no inhibition (i.e., E•I complex has the same activity as E)

and β>1 indicates activation (i.e., E•I complex has more activity than E).

At a given fraction of modified proteasome (f):

v_{c}=k_{c}[20S]ₜ(1−f)+β_{c}k_{c}[20S]ₜ(f) (3)

cₜ=kₜ[20S]ₜ(1−f)+βₜkₜ[20S]ₜ(f) (4)

$$\begin{matrix}Then , \frac{{v}_{c}}{{v}_{t}} = \frac{{k}_{c}}{{k}_{t}} ( \frac{1 - f + {β}_{c} f}{1 - f + {β}_{t} f} ) and & ( 5 ) \\ f = \frac{( \frac{{k}_{c}}{{k}_{t}} - \frac{{v}_{c}}{{v}_{t}} )}{\frac{{k}_{c}}{{k}_{t}} - \frac{{v}_{c}}{{v}_{t}} + {β}_{t} \frac{{v}_{c}}{{v}_{t}} - {β}_{c} \frac{{k}_{c}}{{k}_{t}}} & ( 6 )\end{matrix}$$

The parameter k_{c}/kₜ is an experimentally determinable constant, at least within an individual and possibly across a species. k_{c}/kₜ is dependent on the assay conditions for measurement of the chymotryptic and tryptic activities, but is not dependent on the identity of the inhibitor. The parameters β_{c} and βₜ are constants for a particular inhibitor. Their dependence on assay conditions is expected to be much less than k_{c}/kₜ since the inhibitor-enzyme complex activity must be altered in activity differentially from the free enzyme activity. If f=0 or 1, there is expected to be no dependence of β on assay conditions. Once k_{c}/kₜ, β_{c}, and βₜ are known under a particular set of assay conditions and inhibitor, the chymotryptic and tryptic activities of a crude sample can be used to calculate the fraction of modified proteasome.

In the specific case of N-(pyrazine)carbonyl-L-phenylalanine-L-leucine boronic acid, β_{c}=0, so

$$\frac{{v}_{c}}{{v}_{t}} = \frac{{k}_{c}}{{k}_{t}} ( \frac{1 - f}{1 - f + {β}_{t} f} ) \qquad ( 8 )$$

Analogous equations can be derived for expression of proteasome inhibition as a function of the ratio of any two peptidase activities of the proteasome.

### Example 10

### Assay to measure 20 S proteasome activity in peripheral white blood cells

### Ratio of chymotryptic to tryptic activity

The assay is based upon the SDS-inducible chymotrypsin-like and trypsin-like activities of free 20 S proteasome particles. It uses fluorometry to measure the rate at which the 20 S proteasome hydrolyzes an amide bond in a small peptide substrate. Since some inhibitors of 20 S proteasome activity completely inhibit the chymotrypsin-like activity but activate the trypsin-like activity, the percent of 20 S proteasome bound by such an inhibitor can be directly determined by the ratio of the chymotrypsin-like and trypsin-like activities.

ABBREVIATIONS AND DEFINITIONS:

In addition to the definitions set forth in Example 3, the following definition also applies:

Rs substrate: (N-benzoylvalylglycylarginyl)-7-amino-4-methylcoumarin (Bz-Val-Gly-Arg-AMC) (Bachem)

PROCEDURE

The Ys substrate buffer is prepared as described in Example 3.

The Rs substrate is dissolved to 10 mnM in DMSO. The Rs substrate buffer, containing 20 mM HEPES, 0.5 mM EDTA, 0.6% DMSO, and 60 μM Rs substrate, is prepared.

Purified 20 S proteasome standard from rabbit reticulocytes, prepared according to the literature procedure (McCormack et al., Biochemistry 37:7792-7800 (1998)), is diluted 1:9 (v/v) in 20 mnM HEPES/0.5 mnM EDTA (pH 7.8).

Fluorometer calibration is performed as described in Example 3.

Ys substrate buffer calibration is performed as described in Example 3.

Rs substrate buffer calibration is performed in an analogous fashion, substituting Rs substrate buffer for the Ys substrate buffer.

10 μL of a test sample is added to a cuvette containing 2 mL of Ys substrate buffer at 37° C., and the reaction is allowed to run for 10 minutes. Complete activation of the 20 S proteasome is achieved within 10 minutes. Consistent results are obtained for readings taken after 4 minutes and up to 10 minutes. The maximum linear slope for at least 1 minute of data is measured. If the rate is less than 1 pmol AMC/sec, the measurement is repeated using 20 μL of the test sample.

20 μL of a test sample is added to a cuvette containing 2 mL of Rs substrate buffer at 37° C., and the reaction is allowed to run for 10 minutes. Complete activation of the 20 S proteasome is achieved within 10 minutes. Consistent results are obtained for readings taken after 4 minutes and up to 10 minutes. The maximum linear slope for at least 1 minute of data is measured. If the rate is less than 1 pmol AMC/sec, the measurement is repeated using 20 μL of the test sample in 800 μL Rs buffer.

The percent inhibition (% I) is then calculated according to the following equation:

$$\% I = \frac{100 * ( \frac{{k}_{c}}{{k}_{t}} - \frac{{v}_{c}}{{v}_{t}} )}{( \frac{{k}_{c}}{{k}_{t}} - \frac{{v}_{c}}{{v}_{t}} + {β}_{t} \frac{{v}_{c}}{vt} )} \qquad ( 9 )$$

where v_{c}=(chymotryptic rate (FU/s))/(volume of sample assayed);

vₜ=(tryptic rate (FU/s))/(volume of sample assayed);

k_{c}/kₜ=average v_{c}/vₜ, of 1-3 baseline samples taken from the subject before dosing with the proteasome inhibitor;

βₜ=activation factor determined upon titration of the proteasome inhibitor. For the proteasome inhibitor 1, βₜ=1.28 in human samples.

### Example 11

### Preparation of peripheral whole blood cell lysates for in vitro measurement of 20 S proteasome activity

The required amount of blood is collected into a tube containing anticoagulant. Typically, 1 mL of blood is required. The blood is transferred to a 1.5 mlL Eppendorf microfuge tube and microfuged at 6600 x g for approximately 10 minutes at 4° C. The plasma is aspirated off and the cell pellet is resuspended 1:1 in a volume (˜0.5 mL) of cold phosphate-buffered saline. The cell suspension is again microcentrifuged at 6600 x g for approximately 10 minutes at 4° C. The supernatant is aspirated off. 10 μL of cell pellet is transferred to a 1.5 mL Eppendorf microfuge tube and 0.5 mL of 5 mM EDTA is added. The remaining cell pellet is frozen at −70° C.

10-20 μL of this sample is used in the assay (typical protein concentration is 5 mg/mnL).

### Example 12

### Assay to measure 20 S proteasome activity in peripheral whole blood cells Ratio of chymotryptic-like activity to tryptic-like activity

ABBREVIATIONS AND DEFINITIONS:

The abbreviations and definitions set forth in Examples 3 and 5 apply.

PROCEDURE

The Ys substrate is dissolved to 6 mM in DMSO. A 2% (2 g/100 mL) solution of SDS in MilliQ water is prepared in a glass bottle. The Ys substrate buffer, containing 20 mM HEPES, 0.5 mM EDTA, 0.05% SDS, 1% DMSO, and 60 μM Ys substrate, is prepared. The final pH of the Ys buffer is 8.0.

The Rs substrate is dissolved to 10 mM in DMSO. The Rs substrate buffer, containing 20 mM HEPES, 0.5 mM EDTA, 0.6% DMSO, and 60 μM Rs substrate, is prepared. The final pH of the Rs buffer is 8.0.

Standard whole blood lysate is prepared as described in Example 6 and diluted 1:9 in 20 mM HEPES/0.5 EDTA (pH 7.8).

Fluorometer calibration is performed as described in Example 3, using standard whole blood lysate in place of 20 S proteasome standard.

Ys substrate buffer calibration is performed as described in Example 3, using standard whole blood lysate in place of 20 S proteasome standard.

Rs substrate buffer calibration is performed in an analogous fashion, substituting Rs substrate buffer for the Ys substrate buffer.

A test sample containing 60 μg of protein is added to a cuvette containing 2 mL of Ys substrate buffer at 37° C., and the reaction is allowed to run for 10 minutes. Complete activation of the 20 S proteasome is achieved within 10 minutes. Consistent results are obtained for readings taken after 4 minutes and up to 10 minutes. The maximum linear slope for at least 1 minute of data is measured. If the rate is less than 1 pmol AMC/sec, the measurement is repeated, increasing the amount of the test sample to 120 μg of protein.

A test sample containing 60 μg of protein is added to a cuvette containing 2 mL of Rs substrate buffer at 37° C., and the reaction is allowed to run for 10 minutes. Complete activation of the 20 S proteasome is achieved within 10 minutes. Consistent results are obtained for readings taken after 4 minutes and up to 10 minutes. The maximum linear slope for at least 1 minute of data is measured. If the rate is less than 1 pmol AMC/sec, the measurement is repeated using 120 μg of the test sample.

The percent inhibition (% I) is then calculated according to the following equation:

$$\% I = \frac{100 * ( \frac{{k}_{c}}{{k}_{t}} - \frac{{v}_{c}}{{v}_{t}} )}{( \frac{{k}_{c}}{{k}_{t}} - \frac{{v}_{c}}{{v}_{t}} + {β}_{t} \frac{{v}_{c}}{vt} )} \qquad ( 9 )$$

where v_{c}=(chymotryptic rate (FU/s))/(volume of sample assayed);

vₜ=(tryptic rate (FU/s))/(volume of sample assayed);

k_{c}/kₜ=average vlvt of 1-3 baseline samples taken from the subject before dosing with the proteasome inhibitor;

βₜ=activation factor determined upon titration of the proteasome inhibitor. For the proteasome inhibitor 1, βₜ=1.28 in human samples.

### Example 13

### Proteasome activity levels in peripheral white blood cells of human volunteers

METHODS

Blood samples (approximately 2 mL each) were obtained on five occasions from seven human volunteers over a period of ten weeks. After collection, white blood cells were isolated from the individual blood samples using a Nycoprep^{™}. The resulting pellet was stored in a freezer set to maintain −60° C. to −80° C. until the day of testing. Samples collected on each occasion were tested together and each sample was tested in duplicate.

20 S proteasome activity was determined by measuring the rate of proteolytic hydrolysis of a fluorescent(AMC)-tagged peptide substrate by the sample and normalizing the activity to the amount of protein present in the lysate. 5 μL of sample was added to a cuvette containing 2 mL of assay reaction buffer (20 mM HEPES, 0.5 M EDTA, 0.035% SDS, 60 μM Suc-Leu-Leu-Val-Tyr-AMC in 1.0% DMSO) and magnetic stir bar. The cuvette was placed in a fluorometer and maintained at 37° C. while the amount of hydrolyzed AMC was measured by monitoring the increase in detectable fluorescence over a 5 min period (λem=440 nm; λex=380 nm). A linear regression fit of the reaction progress curve of data collected between 3 and 5 minutes after initiation of the reaction gave the rate of hydrolysis in fluorescent units per second (FU/sec). Protein and hemoglobin concentrations were determined using a modified Bradford assay (Pierce) and a hemoglobin-specific enzymatic-based assay (Sigma), respectively. The total amount of protein measured in the sample was corrected by subtraction of the amount of protein contributed by red blood cells (estimated from the hemoglobin concentration). 20 S proteasome activity in the sample was determined from the equation:

$$20 S proteasome activity ( pmoles AMC / sec / mg protein ) = \frac{( FU / sec ) / ( 5 \times {10}^{- 6} mL ) ( protein μg / mL )}{C}$$

where C=conversion factor equating the amount of fluorescence to the concentration of free AMC (FW/pmole AMC).

RESULTS AND DISCUSSION

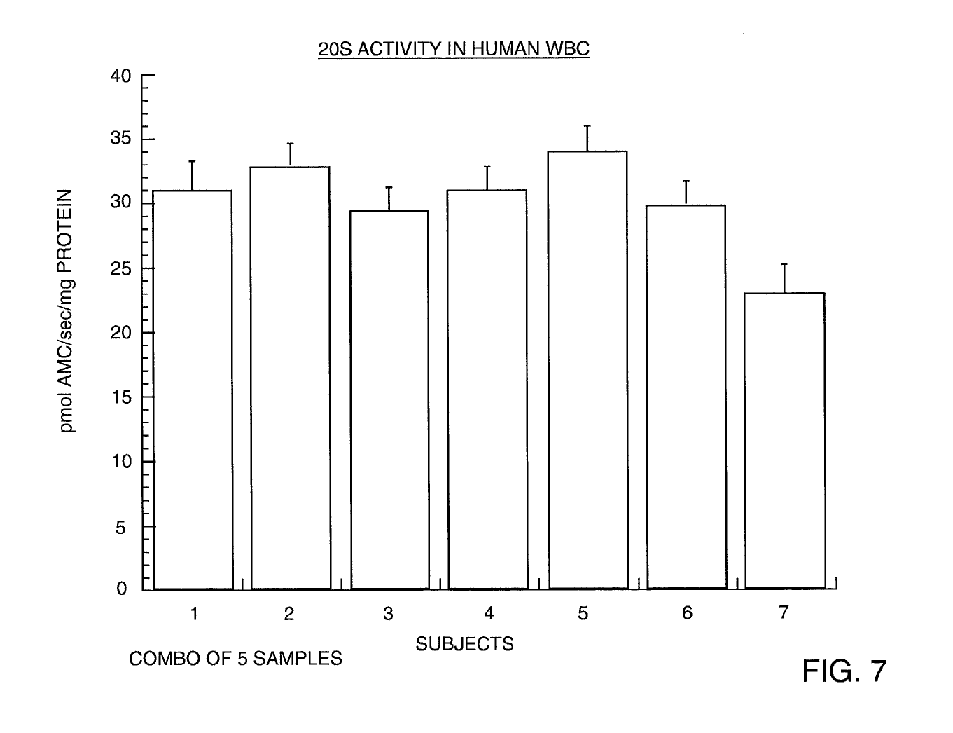

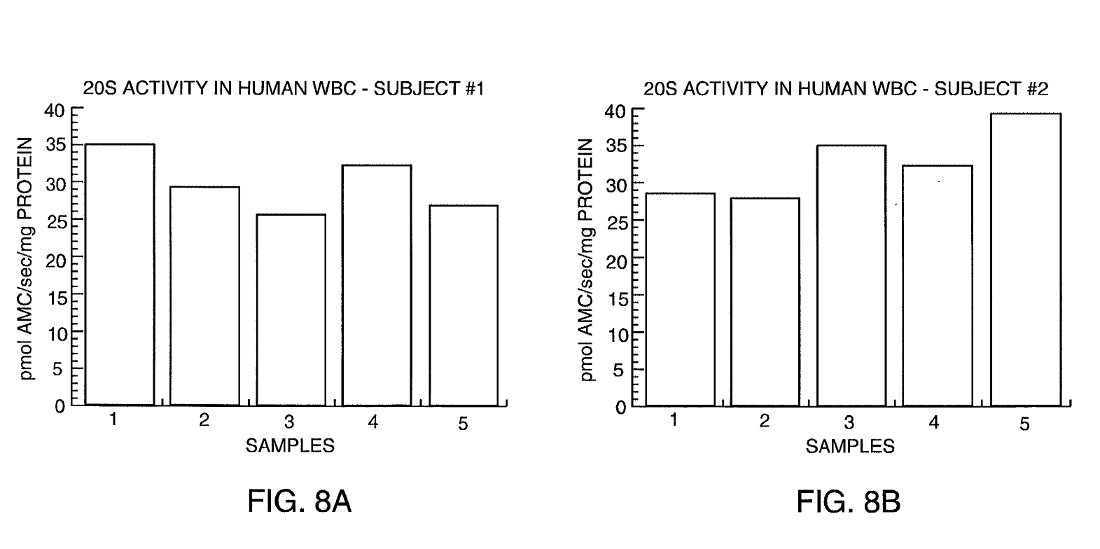

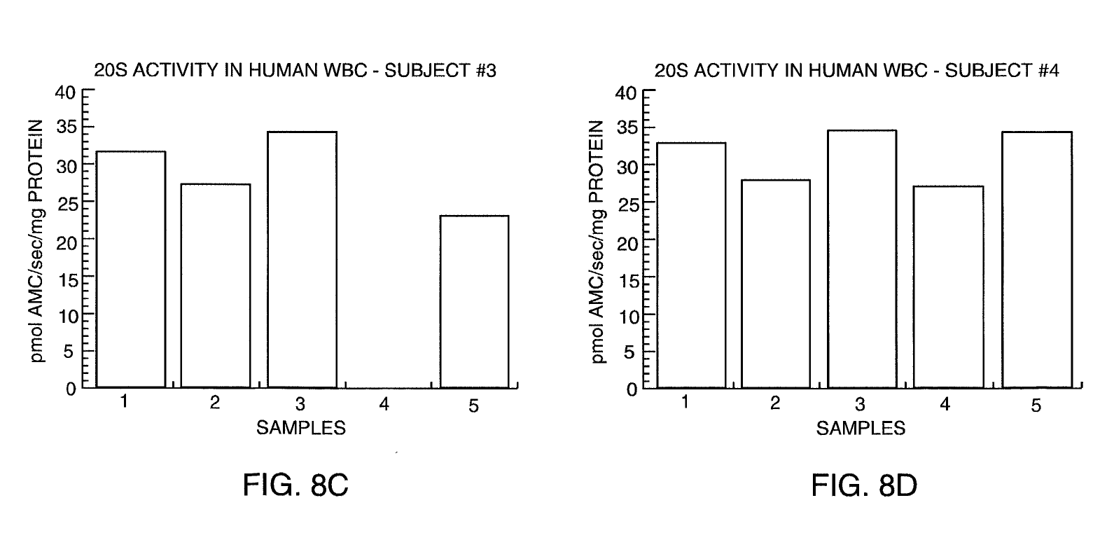

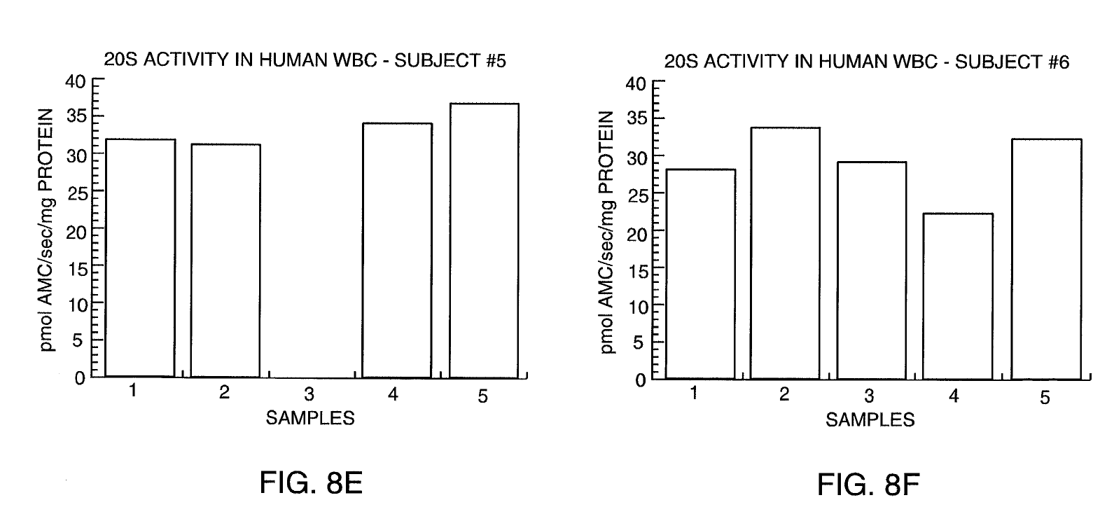

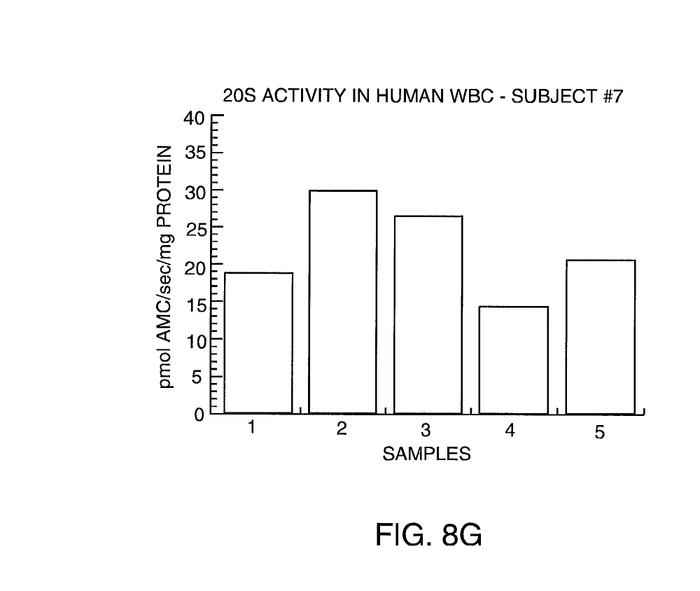

The average 20 S proteasome activity values found for each human volunteer ranged from 15.33 to 40.04 pmol AMC/sec/mg protein (Table 1 and FIG. 7). The activities found across each test day are presented in FIG. 8. The average 20 S proteasome activity found in the population was 29.97+0.80 pmol AMC/sec/mg protein.

TABLE 1
20S Proteasome Activity Levels in Human Volunteers

20S Proteasome Activity
(pmol AMC/sec/mg protein)

| Volunteer | Average ± SEM | Range |
|---|---|---|
| A | 31.05 ± 2.13 | 26.32-35.77 |
| B | 32.77 ± 1.88 | 27.94-40.04 |
| C | 29.33 ± 1.93 | 23.29-34.62 |
| D | 30.90 ± 1.87 | 26.69-34.04 |
| E | 33.91 ± 2.00 | 31.69-37.15 |
| F | 29.66 ± 2.01 | 22.78-34.66 |
| G | 23.07 ± 2.11 | 15.33-31.17 |
| Population | 29.97 ± 0.80 | 15.33-40.04 |
| Average | | |

### Example 14:

### Temporal 20 S Proteasome Activity in Isolated White Blood Cells and Tissues Following Administration of N-(Pyrazine)carbonyl-L phenylalanine-Lleucine Boronic Acid (1)

GENERAL PROCEDURES

Dose formulations of 1 were prepared daily during the course of the study. Dilutions were prepared from a stock solution. The stock solution of 1 was made up in 98% saline (0.9%), 2% ethanol with 0.1% ascorbic acid. Dilutions of the stock were made in the same vehicle.

Female CD2-F1 mice (18 to 20 g), female BALB/c mice (18 to 20 g), female Wistar rats (I 50 to 200 g) and male Sprague-Dawley rats (250 to 450 g) were obtained from Taconic Farms (Germantown, N.Y.). Animals were observed for at least one week and examined for general health before study initiation. Animals used in these studies were asymptomatic. Mice were housed 5 per cage and rats 3 per cage in polycarbonate cages. Corn Cob bedding (AND-1005; Farmers Exchange, Framingham, MA) was used during the observation and study periods. Fluorescent lighting was controlled to automatically provide alternate light and dark cycles of approximately 12 hours each. Temperature and humidity were centrally controlled and recorded daily and readings ranged between 21±2° C. and 45±5%, respectively. Pellets of standard rodent chow (#5001, Purina, St. Louis, Mo.) were available ad libitum throughout the observation and study periods. Cambridge city tap water was provided by water bottles ad libitum. No contaminants of food and water are known which would be expected to interfere with the study.

Drugs were administered in vehicle intravenously (IV) using a dose volume of 100 μL per mouse or 1.0 mL/kg in rats. Control groups were administered with the vehicle (98% saline [0.9%], 2% ethanol, 0.1% ascorbic acid). Animals were dosed with 1 as a single IV bolus given either once or on multiple occasions. Animals exhibiting moribund activity were euthanized with CO2 inhalation.

Following IV dosing with 1, blood was withdrawn at various time points and peripheral white blood cells were isolated.

### Ex Vivo 20 S Proteasome Activity Determined in Peripheral White Blood Cells of Mice After Single Intravenous Administration of 1

In two combined studies, female CD2-F1 mice (18 to 20 g) and female BALB/c mice (18 to 20 g) were administered a single intravenous dose of 1 (0.1 to 3.0 mg/kg in a dose volume of 100 μL). The vehicle was 98% saline [0.9%], 2% ethanol, 0.1% ascorbic acid. Blood samples were collected at 1.0 and 24 h following administration. Due to the blood volume required in the 20 S proteasome activity assay, groups of five mice were sacrificed at the same time and their blood pooled to generate single data points.

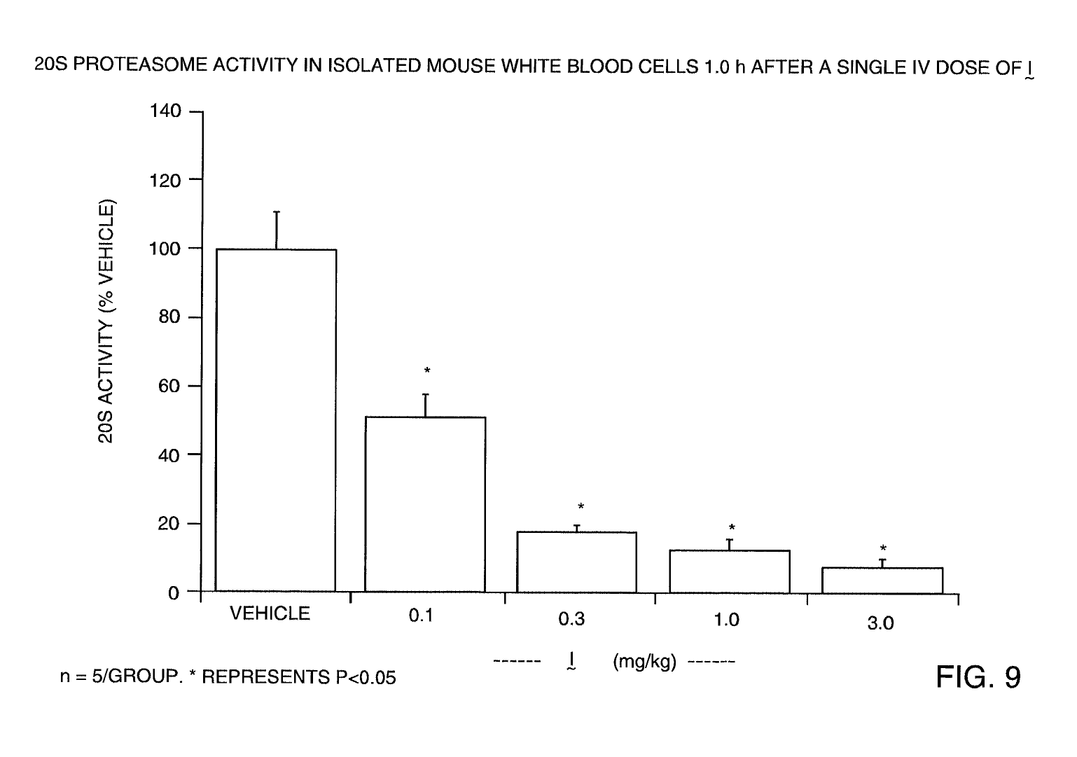

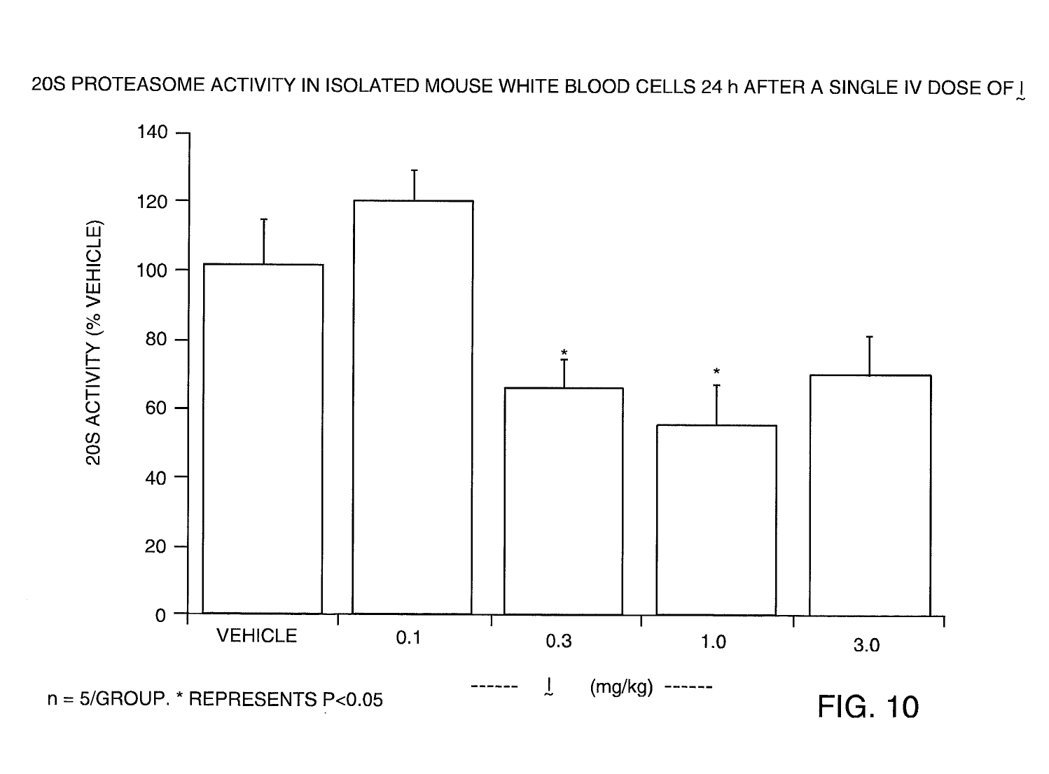

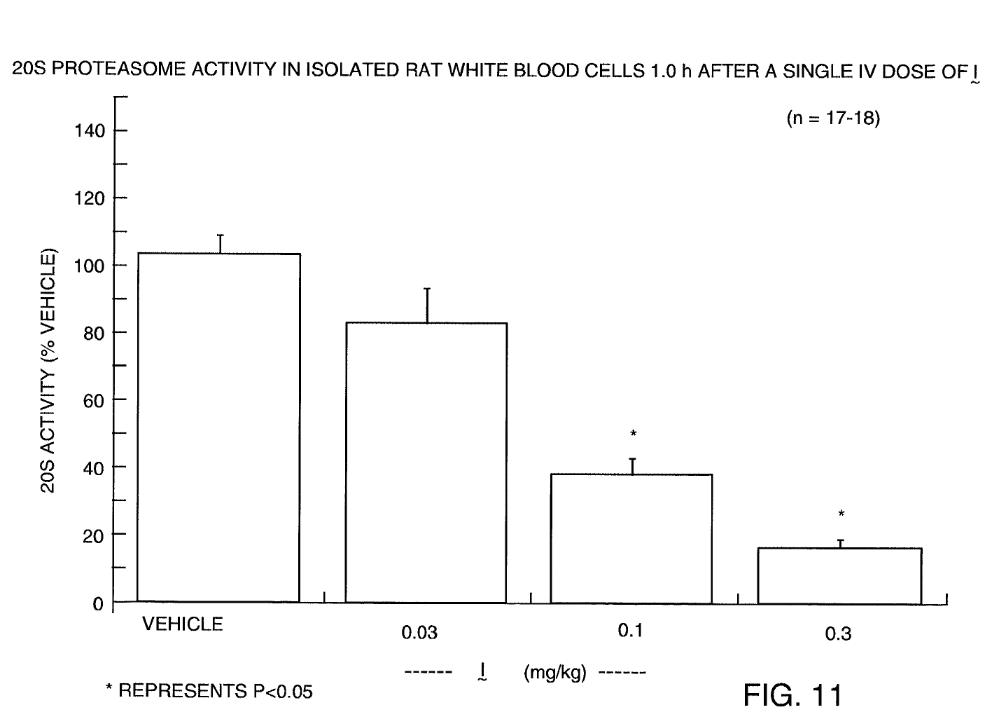

There was a significant (p<0.05) dose-related decrease in 20 S proteasome activity for all dose groups at 1.0 h following intravenous administration of 1 (FIG. 9) which starts to recover at 24 h (FIG. 10). These studies demonstrated a dose-dependent and reversible inhibition of 20 S proteasome activity in the peripheral white blood cells of mice following administration of a single intravenous injection of 1.

### Ex Vivo 20 S Proteasome Activity Determined in Peripheral White Blood Cells of Rats After Single Intravenous Administration of 1

In four combined studies, female Wistar rats (150 to 200 g) were administered a single intravenous dose of 1 (0.03 to 0.3 mg/kg in a dose volume of 1.0 mL/kg). The vehicle was 0.1% ascorbic acid/2% ethanol/98% saline (0.9%). Blood samples were collected at 1.0, 24 and 48 h following administration of 1.

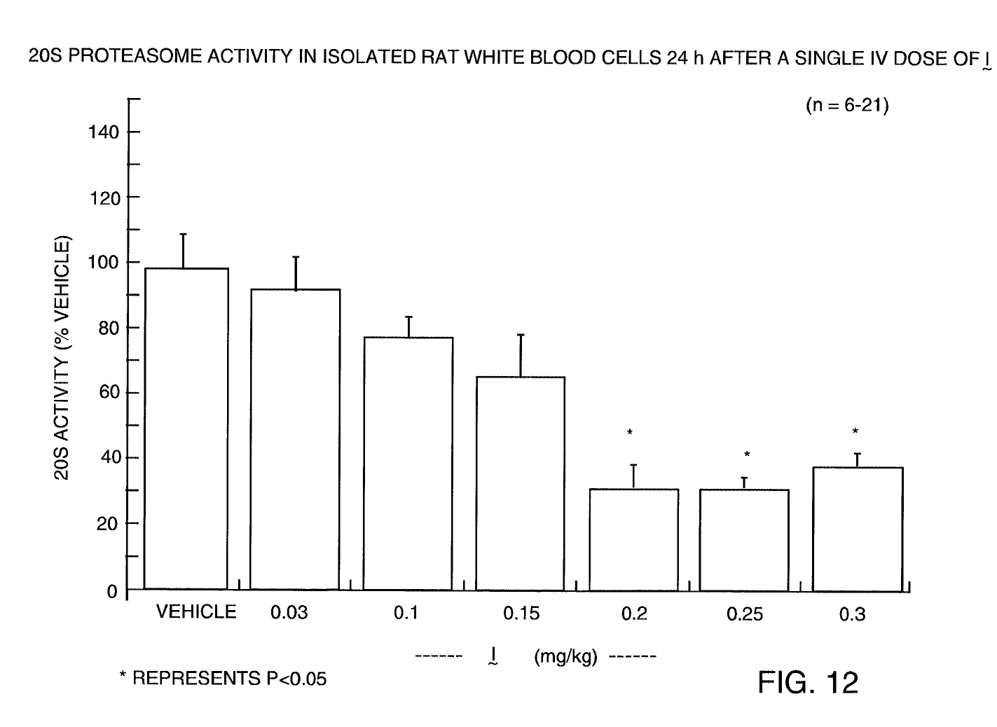

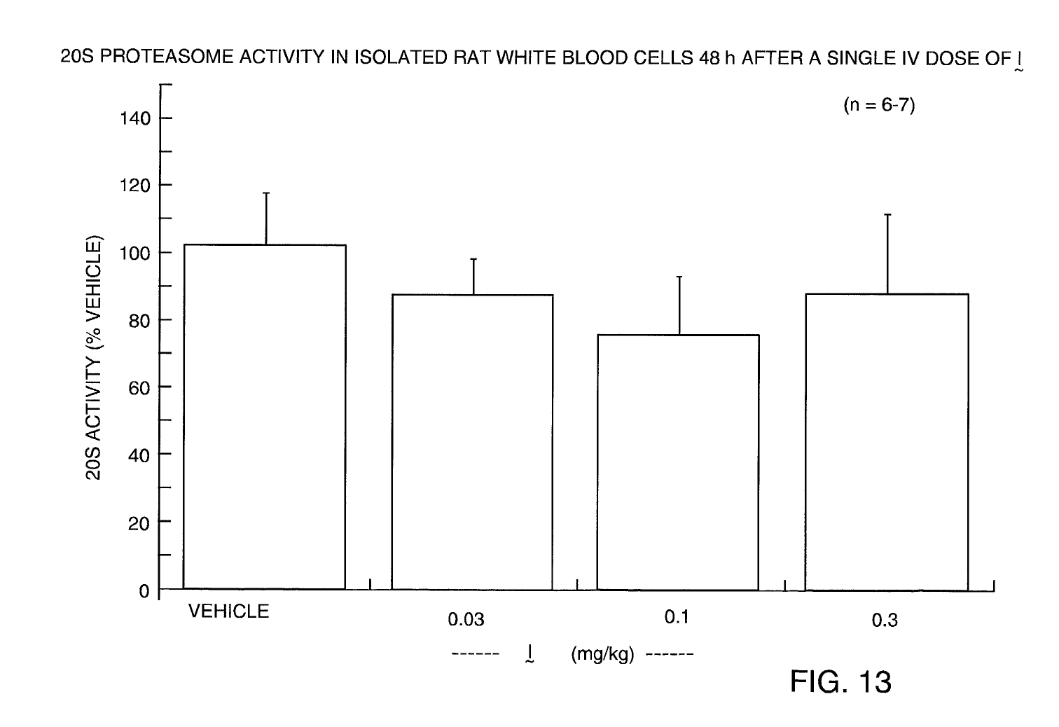

There was a significant (p<0.05) dose-related decrease in 20 S proteasome activity at 1.0 h following intravenous administration of I (FIG. 1 1). Twenty-four hours after administration, the dose-related decreases in 20 S proteasome activity were smaller, but remained significant (p <0.05) in the higher dose groups, ( 0.2 mg/kg (FIG. 12). At 48 h after administration, 20 S proteasome activity was no longer significantly decreased (FIG. 13).

These studies demonstrated a dose-dependent and reversible inhibition of 20 S proteasome activity in the peripheral white blood cells of rats following administration of a single intravenous injection of 1. A slower rate of return to baseline for 20 S proteasome activity levels was observed in rats, possibly indicating faster metabolism of I in mice.

### Ex Vivo 20 S Proteasome Activity Determined in Peripheral White Blood Cells of Rats After Repeat Intravenous Administration of 1

When daily intravenous 1 was administered for 7 days, a dose-related decrease in 20 S proteasome activity was observed 24 h after administration of the last dose. Significant inhibition was observed for doses >0.05 mg/kg. The extent of 20 S proteasome inhibition observed 24 h after administration of 7 daily intravenous doses was greater than that observed 24 h after administration of a single intravenous dose and probably reflects a cumulative effect of daily administration of 1 on its biological target, the proteasome.

A significant dose-related decrease in 20 S proteasome activity was observed 24 h after administration of the last dose for alternate daily intravenous administration of 1 for 14 days. The dose-related decreases in 20 S proteasome activity were significant (p <0.05) in the dose groups >0.2 mg/kg. A significant (p<0.05) dose-related decrease in 20 S proteasome activity was also observed 24 h after administration of the last dose for once weekly intravenous administration of 1 for 8 weeks. The dose-related decreases in 20 S proteasome activity were significant (p<0.05) in the dose groups>0.1 mg/kg.

In an additional repeat dose study, male Sprague-Dawley rats (250 to 450 g; n=6 per group) were treated with twice weekly intravenous doses of 1 (0.01 to 0.35 mg/kg/day in a dose volume of 1.0 mL/kg) for two weeks. The vehicle was 0. 1% ascorbic acid/2% ethanol/98% saline (0.9%). Blood samples were collected 1.0 h after the last dose for evaluation of 20 S proteasome activity.

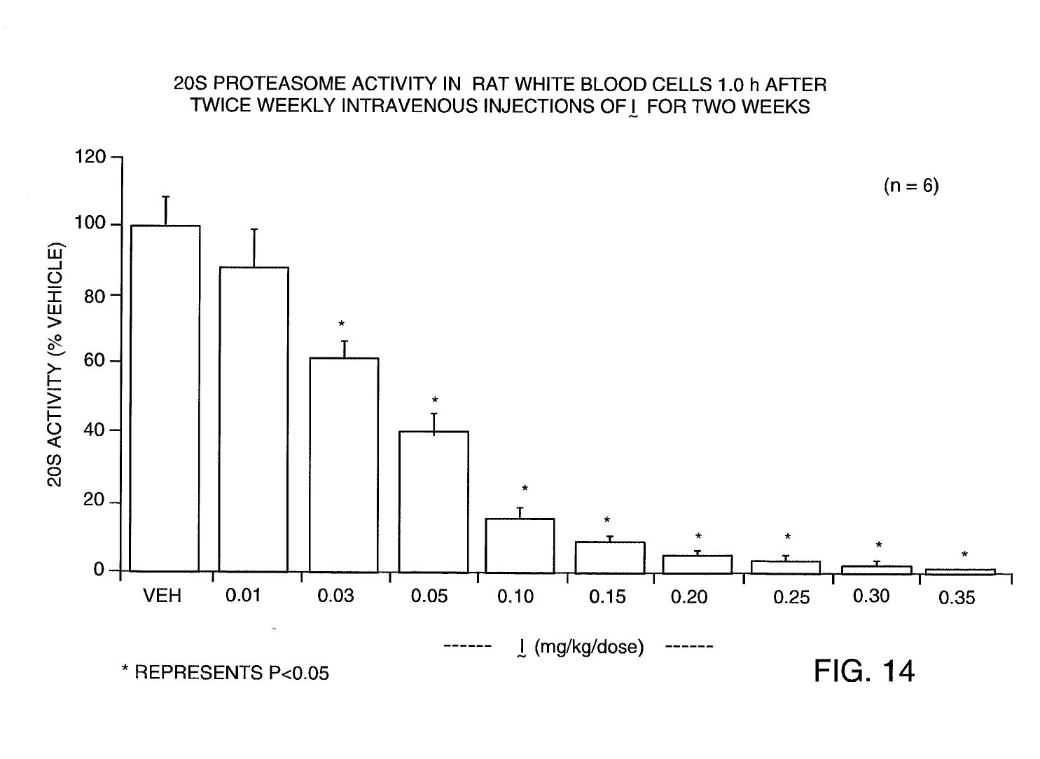

When 1 was administered twice weekly for 2 weeks (total of 4 doses), a dose-related decrease in 20 S proteasome activity was observed 1.0 h after the last dose (FIG. 14). The dose-related decreases in 20 S proteasome activity were significant (p<0.05) for all dose groups>0.03 mg/kg.

The results indicate that repeat dose administration of 1 elicits a dose-related decrease in 20 S proteasome activity in rat white blood cells. The extent of inhibition of 20 S proteasome activity is greater than that seen after a single dose when 1 is given daily or every other day. When the interval between doses of 1 is increased to allow for recovery (i.e., once weekly regimens), the degree of inhibition is equivalent to single administration of 1. This pharmacodynamic profile supports twice weekly dosing with 1, wherein transient inhibition is observed.

### Ex Vivo 20 S Proteasome Activity Determined in Rat Tissues After Repeat Intravenous Administration of 1

In two studies, female Wistar rats (150 to 200 g) were administered a single intravenous dose of 1 (0.03, 0.1 and 0.3 mg/kg in a dose volume of 1.0 mL/kg). The vehicle was 0.1% ascorbic acid/2% ethanol/98% saline (0.9%). Tissue samples were collected from liver and brain at 1.0, 24 and 48 h following administration for evaluation of 20 S proteasome activity.

There was a significant (p<0.05) dose-related decrease in 20 S proteasome activity in rat liver at 1.0 h following intravenous administration of 1. Twenty-four hours after administration, the dose-related decreases in 20 S proteasome activity were smaller, but remained significant (p<0.05) in the high dose group, 0.3 mg/kg. At 48 h after administration, the 20 S proteasome activity in rat liver had returned to baseline. The extent of 20 S proteasome inhibition in the liver returned to baseline levels faster than that observed for peripheral white blood cells. No 20 S proteasome inhibition was observed in brain tissue, reflecting the lack of penetration of 1 into this tissue.

In a third study, male Sprague-Dawley rats (250 to 450 g) were administered a single intravenous dose of 1 (0.1 and 0.3 mg/kg in a dose volume of 1.0 mL/kg). The vehicle was 0.1% ascorbic acid/2% ethanol/98% saline (0.9%). Blood and tissue samples were collected 1.0 h following administration for evaluation of 20 S proteasome activity. The tissues collected were brain, colon, liver, muscle (gastrocnemius), prostate and testes.

Significant (p<0.05) dose-related decreases in 20 S proteasome activity were observed in peripheral white blood cells, colon, liver, muscle (gastrocnemius), and prostate at 1.0 h following intravenous administration of 1. No 20 S proteasome inhibition was observed in brain and testes, reflecting the lack of 1 penetration into these tissues.

The 20 S proteasome inhibition in tissues 1.0 h after intravenous dose administration, except for brain and testes, was similar to that observed for peripheral white blood cells.

### Ex Vivo 20 S Proteasome Activity Determined in Primates After Single Intravenous Administration of 1

Male and female Cynomolgus monkeys (2.2 to 3.5 kg) were assigned to four groups (5/sex/group). Each group received 0 (vehicle control), 0.045, 0.067 or 0.100 mg/kg/dose of 1 as a single intravenous injection in a dose volume of 0.3 mL/kg twice weekly for 4 weeks (days 1, 5, 8, 12, 15, 19, 22 and 26). The vehicle was 0.1% ascorbic acid/2% ethanol/98% saline (0.9%). Three males from the control, low- and mid-dose groups, two high-dose males, and three females/group were sacrificed at the end of treatment on Day 27. Two animals/sex/group were designated as recovery animals and received treatment for 4 weeks followed by 2 weeks of recovery; they were sacrificed on Day 41.

Blood was collected for 20 S proteasome activity determination prior to treatment, at 1.0 h after dosing on Days 1, 8, 15 and 22, and at 1.0 h prior to dosing on Days 5, 12, 19 and 26; and on Days 31, 34, 38, and 41 (recovery sacrifice animals). Blood was also collected for 20 S proteasome activity determination from the high-dose male before it was sacrificed in moribund condition on Day 26 after receiving 8 doses.

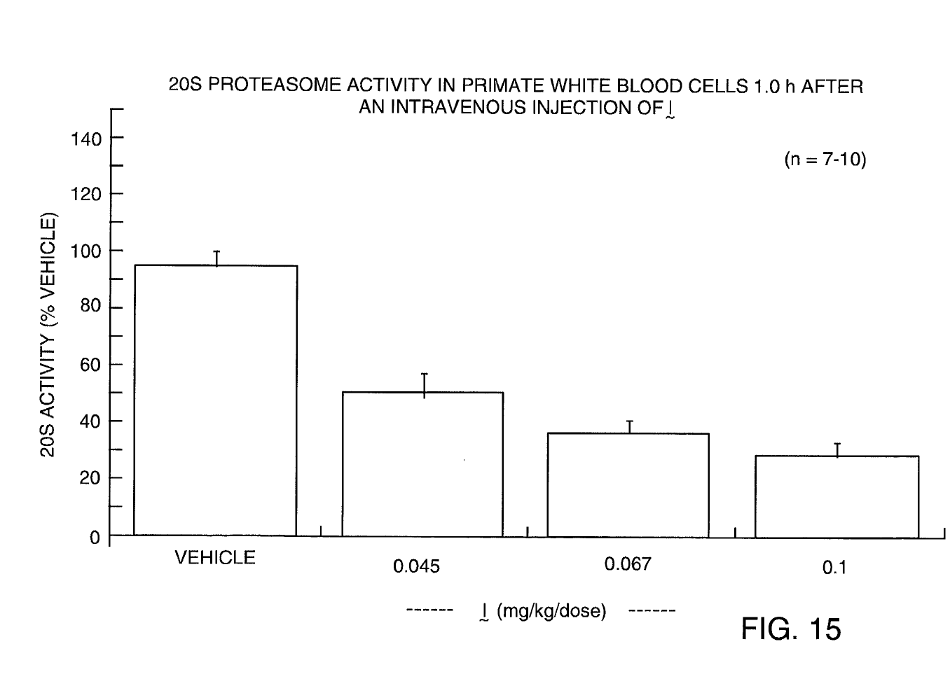

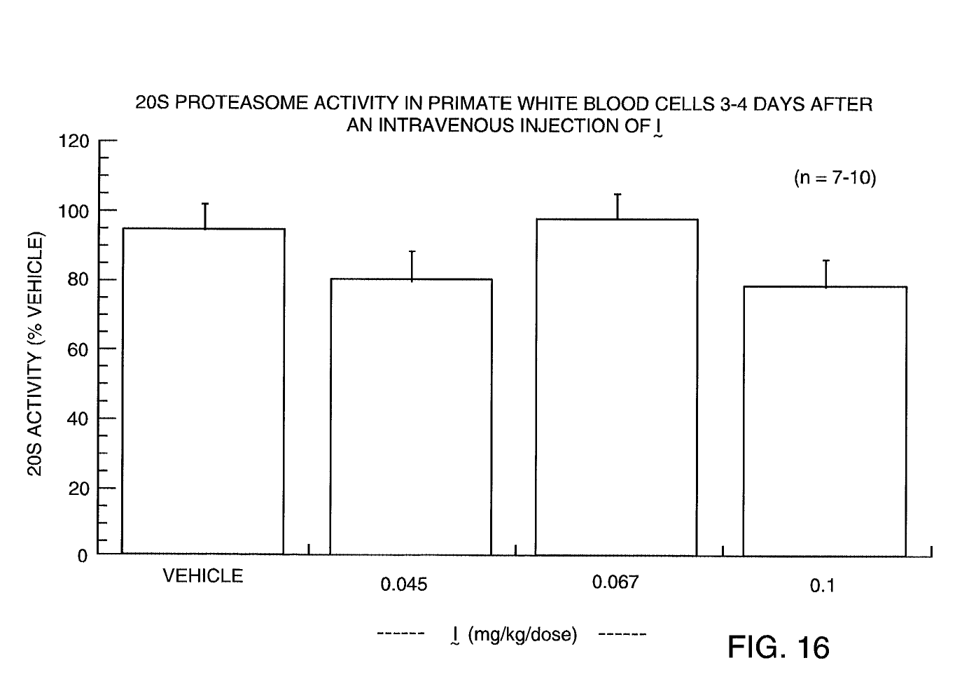

Determination of white blood cell 20 S proteasome activity 1.0 h after dosing revealed a significant and dose-related decrease in enzyme activity that had recovered by 72 hours, prior to the subsequent dose (FIGS. 15 and 16). The moribund animal was found to have low residual 20 S proteasome activity in its white blood cells at sacrifice on Day 26.

These data support a twice weekly treatment regimen for 1, since the 20 S proteasome levels recover between doses.

### Example 15

### Effect of N-(pyrazine)carbonyl-Lphenylalanine-Ileucine boronic acid (1) on the Chymotryptic and Tryptic Activities of Puiifted 20 S Proteasome from Rabbit Reticulocytes

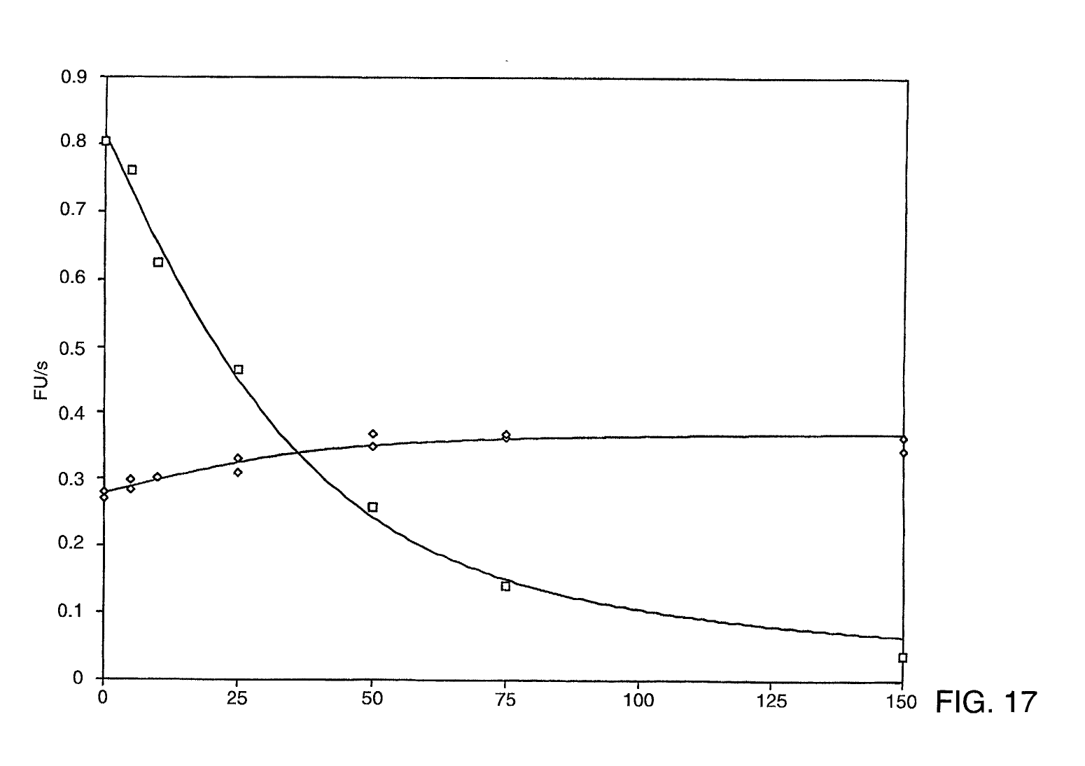

20 S Proteasome was purified from rabbit reticulocytes according to published procedures (McCormack et al., Biochemistry 37:7792-7800 (1998)). Chymotryptic and tryptic assays were performed as described in Examples 3 and 5 at increasing concentrations of the proteasome inhibitor 1. Data is presented in FIG. 17.

### Example 16

### Correlation of Percent Inhibition and Ratio of Chymotryptic Activity to Tryptic Activity in Purified 20 S Proteasome from Rabbit Reticulocytes

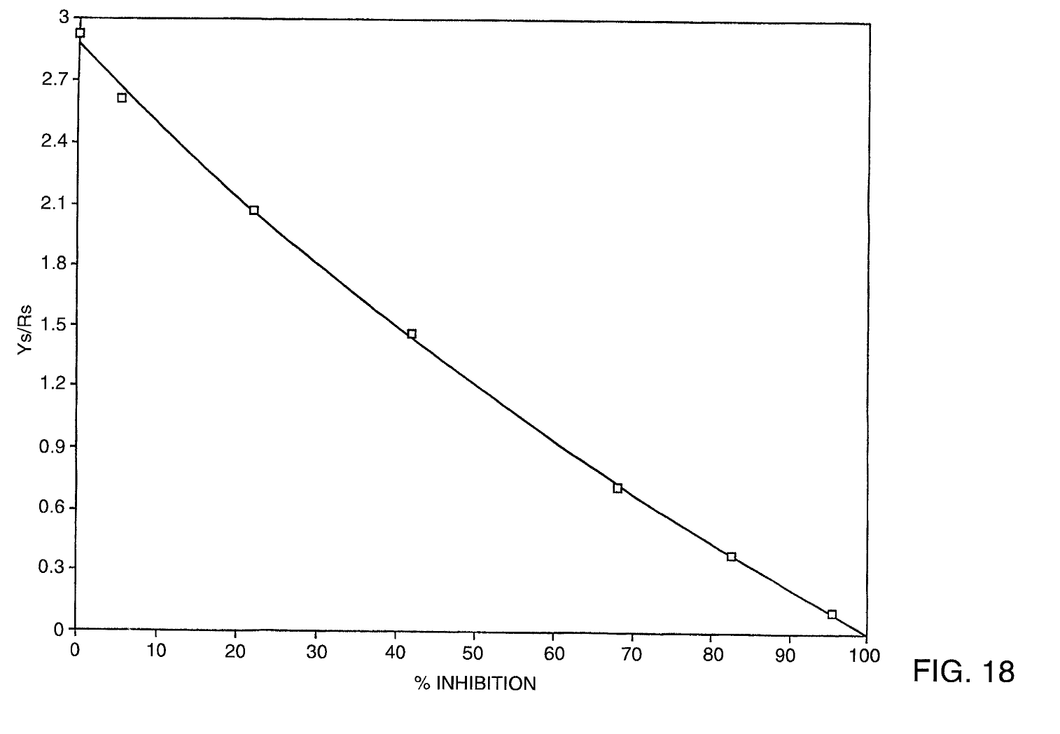

Purified 20 S proteasome from rabbit reticulocytes was prepared according to published procedures (McCormack et al., Biochemistry 37:7792-7800 (1998)). Chymotryptic and tryptic assays were performed as described in Examples 3 and 5 at increasing concentrations of the proteasome inhibitor 1. The data was fitted to vc/vt =kc/kt*(1−f)/(1−f+βt*f), where kc/kt=2.88±0.03, βt=1.38±0.05, and % I=f*100 (FIG. 18).

### Example 17

### Correlation of Percent Inhibition and Ratio of Chymotryptic Activity to Tryptic Activity in Rat White Blood Cell Lysates

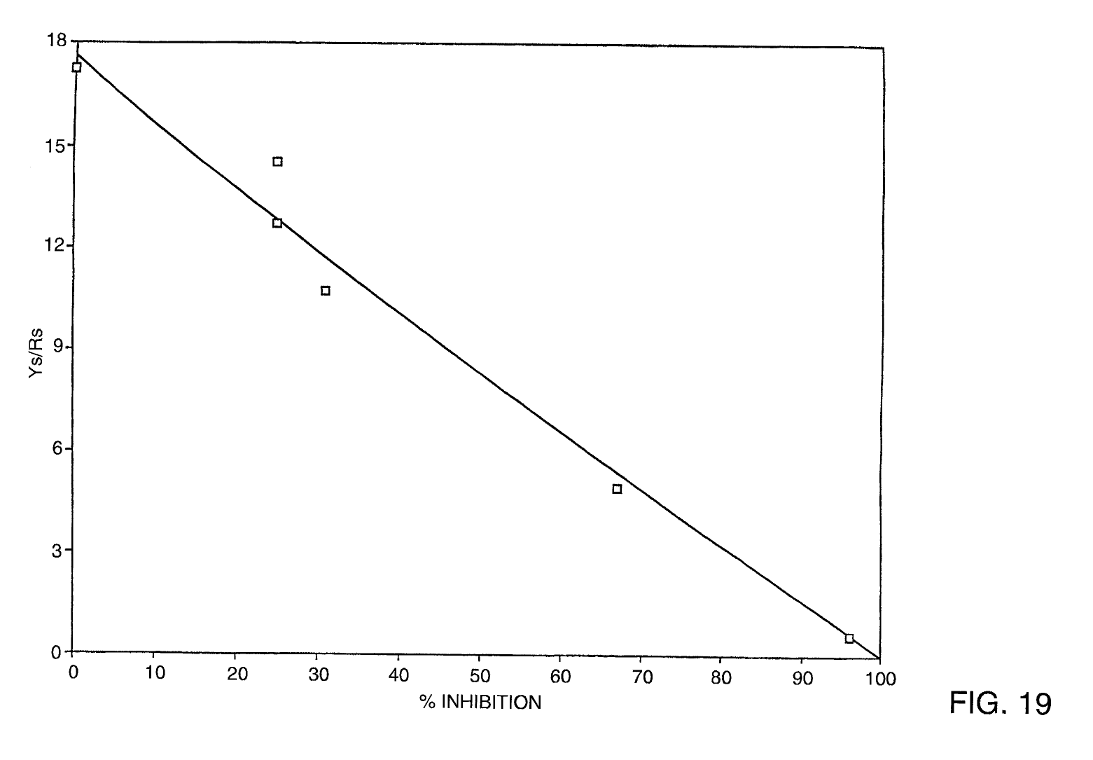

Chymotryptic and tryptic assays were performed as described in Examples 3 and 5 at increasing concentrations of the proteasome inhibitor 1. Data was fitted as in Example 10, to give kc/kt=17.6±and βt=1.1±0.2 (FIG. 19).

What is claimed is:

1. A method for treating a patient afflicted with multiple sclerosis comprising administering to the patient an effective amount of an agent selected from the group consisting of proteasome inhibitors, ubiquitin pathway inhibitors, agents that interfere with the activation of NF-κB via the ubiquitin proteasome pathway, and mixtures thereof.

2. The method according to claim 1, wherein the agent is administered in an amount sufficient to reduce the frequency or severity of relapse.

3. The method according to claim 1, wherein the agent is a proteasome inhibitor.

4. The method according to claim 3, wherein the proteasome inhibitor is lactacystin or a lactacystin analog compound.

5. The method according to claim 4, wherein the lactacystin analog compound is selected from the group consisting of lactacystin, clasto-lactacystin β-lactone, 7-ethyl-clasto-lactacystin β-lactone, 7-n-propyl-clasto-lactacystin β-lactone, and 7-n-butyl-clasto-lactacystin β-lactone.

6. The method according to claim 5, wherein the lactacystin analog compound is 7-n-propyl-clasto-lactacystin β-lactone.

7. A method for treating a patient afflicted with asthma comprising administering to the patient an effective amount of an agent selected from the group consisting of proteasome inhibitors, ubiquitin pathway inhibitors, agents that interfere with the activation of NF-κB via the ubiquitin proteasome pathway, and mixtures thereof.

8. The method according to claim 7, wherein the agent is administered in an amount sufficient to reduce the frequency or severity of asthmatic attack.

9. The method according to claim 7, wherein the agent is a proteasome inhibitor.

10. The method according to claim 9, wherein the proteasome inhibitor is lactacystin or a lactacystin analog compound.

11. The method according to claim 10, wherein the lactacystin analog compound is selected from the group consisting of lactacystin, clasto-lactacystin β-lactone, 7-ethyl-clasto-lactacystin β-lactone, 7-n-propyl-clasto-lactacystin β-lactone, and 7-n-butyl-clasto-lactacystin β-lactone.

12. The method according to claim 11, wherein the lactacystin analog compound is 7-n-propyl-clasto-lactacystin β-lactone.

13. A method for treating a patient afflicted with asthma comprising administering to the patient an effective combination of a glucocorticoid and an agent selected from the group consisting of proteasome inhibitors, ubiquitin pathway inhibitors, agents that interfere with the activation of NF-κB via the ubiquitin proteasome pathway, and mixtures thereof.

14. The method according to claim 13, wherein the combination is administered in an amount sufficient to reduce the frequency or severity of asthmatic attack.

15. The method according to claim 13, wherein the glucocorticoid and the agent are administered at the same time.

16. The method according to claim 13, wherein the glucocorticoid and the agent are administered at different times.

17. The method according to claim 13, wherein the combination comprises an amount of the glucocorticoid that is less than its standard recommended dosage.

18. The method according to claim 13, wherein the combination comprises an amount of the agent sufficient to reduce the dose or treatment frequency required for the glucocorticoid.

19. The method according to claim 13, wherein the combination comprises an amount of the glucocorticoid sufficient to reduce the dose or treatment frequency required for the agent.

20. The method according to claim 13, wherein the agent is a proteasome inhibitor.

21. The method according to claim 20, wherein the proteasome inhibitor is lactacystin or a lactacystin analog compound.

22. The method according to claim 21, wherein the lactacystin analog compound is selected from the group consisting of lactacystin, clasto-lactacystin β-lactone, 7-ethyl-clasto-lactacystin β-lactone, 7-n-propyl-clasto-lactacystin β-lactone, and 7-n-butyl-clasto-lactacystin β-lactone.

23. The method according to claim 22, wherein the lactacystin analog compound is 7-n-propyl-clasto-lactacystin β-lactone.

24. The method according to claim 13, wherein the glucocorticoid is selected from the group consisting of flunisolide, triamcinolone acetonide, beclomethasone dipropionate, dexamethasone sodium phosphate, fluticasone propionate, budesonide, hydrocortisone, prednisone, prednisolone, mometasone, tipredane, and butixicort.

25. The method according to claim 24, wherein the glucocorticoid is budesonide.

26. The method according to claim 13, wherein the agent is 7-n-propyl-clasto-lactacystin β-lactone and the glucocorticoid is budesonide.

27. A pharmaceutical composition comprising an effective combination of a glucocorticoid and an agent selected from the group consisting of proteasome inhibitors, ubiquitin pathway inhibitors, agents that interfere with the activation of NF-κB via the ubiquitin proteasome pathway, and mixtures thereof.

28. The composition of claim 27, wherein said composition is provided in a unit dosage form.

29. The composition of claim 28, wherein the unit dosage form comprises an amount of the glucocorticoid that is less than its standard recommended dosage.

30. The composition of claim 27, wherein said composition comprises the agent in an amount sufficient to reduce the dose or treatment frequency required for the glucocorticoid.

31. The composition of claim 27, wherein the agent is a proteasome inhibitor.

32. The composition of claim 31, wherein the proteasome inhibitor is lactacystin or a lactacystin analog compound.

33. The composition of claim 32, wherein the lactacystin analog compound is selected from the group consisting of lactacystin, clasto-lactacystin β-lactone, 7-ethyl-clasto-lactacystin β-lactone, 7-n-propyl-clasto-lactacystin β-lactone, and 7-n-butyl-clasto-lactacystin β-lactone.

34. The composition according to claim 33, wherein the lactacystin analog compound is 7-n-propyl-clasto-lactacystin β-lactone.

35. The composition according to claim 27, wherein the glucocorticoid is selected from the group consisting of flunisolide, triamcinolone acetonide, beclomethasone dipropionate, dexamethasone sodium phosphate, fluticasone propionate, budesonide, hydrocortisone, prednisone, prednisolone, mometasone, tipredane, and butixicort.

36. The composition according to claim 35, wherein the glucocorticoid is budesonide.

37. The composition according to claim 27, wherein the agent is 7-n-propyl-clasto-lactacystin β-lactone and the glucocorticoid is budesonide.
